(19) United States
(12) Patent Application Publication
(10) Pub. No.: US 2006/0142202 A1
(43) Pub. Date: Jun. 29, 2006

(54) Compositions and methods for targeted delivery of immune response modifiers

(75) Inventors: Sefik Alkan, Woodbury, MN (US); William C. Kieper, Stillwater, MN (US); John P. Vasilakos, Woodbury, MN (US); Jason D. Bonk, Hudson, WI (US); George W. Griesgraber, Eagan, MN (US); Kenneth E. Lipson, Lakeland Shores, MN (US); Jie J. Liu, Woodbury, MN (US); James D. Mendoza, Robbinsdale, MN (US); Doris Stoermer, White Bear Lake, MN (US); Paul D. Wightman, Woodbury, MN (US); Naiyong Jing, Woodbury, MN (US); William J. Schultz, North Oaks, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/360,071

(22) Filed: Feb. 23, 2006

Related U.S. Application Data
(63) Continuation-in-part of application No. 11/220,235, filed on Sep. 6, 2005.
Application No. 11/220,235 is a continuation of application No. 10/013,193, filed on Dec. 6, 2001, now abandoned.
(60) Provisional application No. 60/254,229, filed on Dec. 8, 2000.

(51) Int. Cl. A61K 38/09; A61K 31/4745

(52) U.S. Cl. 514/15; 514/292

(57) ABSTRACT

The present invention provides immunomodulatory compositions include an immune response modifier moiety coupled to a targeting moiety. In another aspect, the invention provides methods of providing targeted delivery of an IRM, generating a localized immune response, and treating a condition in a subject. Generally, the methods include administering to the subject an immunomodulatory composition that includes an immune response modifier moiety coupled to a targeting moiety that recognizes a delivery target.

### CROSS-REFERENCE TO RELATED APPLICATIONS

This application is a CIP of U.S. patent application Ser. No. 11/220,235, filed Aug. 6, 2005, which is a continuation of U.S. patent application Ser. No. 10/013,193, filed Dec. 6, 2001, now abandoned, which claims priority to U.S. Provisional Patent Application Ser. No. 60/254,229, filed Dec. 28, 2000. In addition, this application claims priority to U.S. Provisional Application Ser. No. 60/655,713 filed Feb. 23, 2005 and U.S. Provisional Patent Application entitled, “Immune Response Modifier Conjugates,” filed Feb. 22, 2006.

### BACKGROUND

There has been a major effort in recent years, with significant success, to discover new drug compounds that act by stimulating certain key aspects of the immune system, as well as by suppressing certain other aspects (see, e.g., U.S. Pat. Nos. 6,039,969 and 6,200,592). These compounds, referred to herein as immune response modifiers (IRMs), appear to act through basic immune system mechanisms known as Toll-like receptors (TLRs) to induce selected cytokine biosynthesis. They may be useful for treating a wide variety of diseases and conditions. For example, certain IRMs may be useful for treating viral diseases (e.g., human papilloma virus, hepatitis, herpes), neoplasias (e.g., basal cell carcinoma, squamous cell carcinoma, actinic keratosis, melanoma), and T_{H}2-mediated diseases (e.g., asthma, allergic rhinitis, atopic dermatitis), and are also useful as vaccine adjuvants.

Immune response modifiers include compounds that possess potent immunomodulating activity including but not limited to antiviral and antitumor activity. Certain IRMs modulate the production and secretion of cytokines. For example, certain IRM compounds induce the production and secretion of cytokines such as, e.g., Type I interferons, TNF-α, IL-1, IL-6, IL-8, IL-10, IL-12, MIP-1, and/or MCP-1. As another example, certain IRM compounds can inhibit production and secretion of certain T_{H}² cytokines, such as IL-4 and IL-5. Additionally, some IRM compounds are said to suppress IL-1 and TNF (U.S. Pat. No. 6,518,265).

Certain IRMs are small organic molecules (e.g., molecular weight under about 1000 Daltons, preferably under about 500 Daltons, as opposed to large biological molecules such as proteins, peptides, nucleic acids, and the like) such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; and 6,818,650; U.S. Patent Publication Nos. US2004/0091491; US2004/0147543; and US2004/0176367; and International Publication Nos. WO2005/18551, WO2005/18556, WO2005/20999, WO2005/032484, WO2005/048933, WO2005/048945, WO2005/051317, WO2005/051324, WO2005/066169, WO2005/066170, WO2005/066172, WO2005/076783, and WO2005/079195.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), certain 3-β-D-ribofuranosylthiazolo[4,5-d]pyrimidine derivatives (such as those described in U.S. Publication No. US2003/0199461), and certain small molecule immuno-potentiator compounds such as those described, for example, in US2005/0136065.

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304.

Other IRMs include biological molecules such as aminoalkyl glucosaminide phosphates (AGPs) and are described, for example, in U.S. Pat. Nos. 6,113,918; 6,303,347; 6,525,028; and 6,649,172.

The immunostimulatory effects of IRMs may be increased by co-delivery of an IRM compound and an antigen to cells of the immune system. Co-delivery may be accomplished by, for example, covalent or non-covalent chemical coupling of the IRM and antigen, or physically confining the IRM and antigen to a defined space. Methods for co-delivery of IRM and an antigen are described, for example, in U.S. Patent Publication No. US2004/0091491.

In view of the great therapeutic potential for IRMs, and despite the important work that has already been done, there is a substantial ongoing need to expand their uses and therapeutic benefits.

### SUMMARY

It has been found that an immune response modifier material can be coupled to a target-specific material and each portion can retain its respective function. When administered to a subject, the targeting moiety of the resulting immunomodulatory composition can provide targeted delivery of the immune response modifier moiety.

Accordingly, in one aspect, the present invention provides an immunomodulatory composition that includes an immune response modifier moiety coupled to a targeting moiety.

In another aspect, the present invention also provides method of targeted delivery of an immune response modifier compound. Generally, the method includes administering to a subject an immunomodulatory composition that includes an immune response modifier moiety coupled to a targeting moiety that recognizes a delivery target.

In another aspect, the present invention also provides a method of inducing a localized immune response. Generally, the method includes administering to a subject an immunomodulatory composition that includes an immune response modifier moiety coupled to a targeting moiety that recognizes a delivery target in an amount effective to induce an immune response.

In yet another aspect, the present invention provides a method of treating a condition in a subject that is treatable by inducing an immune response. Generally, the method includes administering to the subject an immunomodulatory composition that includes an immune response modifier moiety coupled to a targeting moiety that recognizes a delivery target in an amount effective to treat at least one symptom or sign of the condition.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1 is a line graph that demonstrates anti-CD20 activity of an IRM/anti-CD20 antibody immunomodulatory composition.

FIG. 2 is a line graph that demonstrates anti-CD20 activity of a control compound/anti-CD20 antibody composition.

FIG. 3 is a line graph that demonstrates anti-CD20 activity of a control compound/anti-CD20 antibody composition.

FIG. 4 is a line graph that demonstrates anti-CD20 activity of an IRM/anti-CD20 antibody immunomodulatory composition.

FIG. 5 is a line graph that demonstrates anti-CD20 activity of an IRM/anti-CD20 antibody immunomodulatory composition.

FIG. 6 is a line graph that demonstrates anti-CD20 activity of an IRM/anti-CD20 antibody immunomodulatory composition.

FIG. 7 is a bar graph showing cytokine induction by IRM/anti-CD20 antibody immunomodulatory compositions.

FIG. 8 is a line graph demonstrating anti-CD40 activity of an IRM/anti-CD40 antibody immunomodulatory composition.

FIG. 9 is a line graph demonstrating anti-CD40 activity of an IRM/anti-CD40 antibody immunomodulatory composition.

FIG. 10 is a line graph demonstrating anti-CD40 activity of an IRM/anti-CD40 antibody immunomodulatory composition.

FIG. 11 is a bar graph demonstrating cytokine induction by IRM/anti-CD40 antibody immunomodulatory compositions.

FIG. 12 is a line graph demonstrating anti-CD8 activity of an IRM/anti-CD8 antibody immunomodulatory composition.

FIG. 13 is a line graph demonstrating anti-CD8 activity of an IRM/anti-CD8 antibody immunomodulatory composition.

FIG. 14 is a line graph demonstrating anti-CD8 activity of an IRM/anti-CD8 antibody immunomodulatory composition.

FIG. 15 is a bar graph demonstrating cytokine induction by IRM/anti-CD8 antibody immunomodulatory compositions.

FIG. 16 is a line graph demonstrating anti-HER2 activity of an IRM/anti-HER2 antibody immunomodulatory composition.

FIG. 17 is a line graph demonstrating anti-HER2 activity of an IRM/anti-HER2 antibody immunomodulatory composition.

FIG. 18 is a bar graph demonstrating cytokine induction by IRM/anti-HER2 antibody immunomodulatory compositions.

FIG. 19 is a line graph demonstrating anti-HER2 activity of an IRM/anti-HER2 antibody immunomodulatory composition.

FIG. 20 is a line graph showing IFN-α induction by an IRM/anti-HER2 antibody immunomodulatory composition.

FIG. 21 is a line graph showing TNF-α induction by an IRM/anti-HER2 antibody immunomodulatory composition.

FIG. 22 is a line graph showing IFN-α induction by an IRM/anti-HER2 antibody immunomodulatory composition.

FIG. 23 is a line graph showing TNF-α induction by an IRM/anti-HER2 antibody immunomodulatory composition.

FIG. 24 a line graph that demonstrates the immunospecificity of an IRM/anti-CD8 antibody immunomodulatory composition.

FIG. 25 is a line graph that shows induction of IFN-α in peripheral blood mononuclear cells (PBMCs) by an IRM/anti-CD8 antibody immunomodulatory composition.

FIG. 26 is a line graph that shows induction of TNF-α in PBMCs by an IRM/anti-CD8 antibody immunomodulatory composition.

### DETAILED DESCRIPTION OF ILLUSTRATIVE EMBODIMENTS OF THE INVENTION

The invention provides immunomodulatory compositions in which an immune response modifier (IRM) moiety is coupled to a targeting moiety for targeted delivery of the IRM moiety. Thus, even though the composition may be administered systemically, the targeting moiety can direct, sequester, retain, or otherwise actively target delivery of the IRM moiety, thereby concentrating the IRM moiety at the target site. Concentrating the IRM moiety at a target site may result in various benefits such as, for example, reducing the amount of the IRM moiety that is available systemically, thereby reducing—perhaps even eliminating—systemic side effects associated with administration of the IRM moiety. Also, because the IRM moiety is concentrated at the target site, a smaller dose of the IRM moiety—at least as compared to an uncoupled form of the IRM moiety (i.e., the uncoupled IRM compound)—may be needed to provide effective treatment, which may provide cost and resource benefits as well as further limit the extent, severity, and/or duration of undesirable side effects.

For the purposes of the present invention, the following terms shall have the indicated meanings:

“Agonist” refers to a compound that can combine with a receptor (e.g., a TLR) to induce a cellular activity. An agonist may be a ligand that directly binds to the receptor. Alternatively, an agonist may combine with a receptor indirectly by, for example, (a) forming a complex with another molecule that directly binds to the receptor, or (b) otherwise results in the modification of another compound so that the other compound directly binds to the receptor. An agonist may be referred to as an agonist of a particular TLR (e.g., a TLR6 agonist) or a particular combination of TLRs (e.g., a TLR 7/8 agonist—an agonist of both TLR7 and TLR8).

“Ameliorate” refers to any reduction in the extent, severity, frequency, and/or likelihood of a symptom or clinical sign characteristic of a particular condition.

“Antigen” refers to any substance that may be bound by an antibody in a manner that is immunospecific to some degree.

“Immune cell” refers to cell of the immune system, i.e., a cell directly or indirectly involved in the generation or maintenance of an immune response, whether the immune response is innate, acquired, humoral, or cell-mediated.

“Immunomodulatory” and variations thereof refer to any increase or decrease (i.e., induction or inhibition) of immune activity.

“Induce” and variations thereof refer to any measurable increase in cellular activity. For example, induction of an immune response may include, for example, an increase in the production of a cytokine, activation, proliferation, or maturation of a population of immune cells, and/or other indicator of increased immune function.

“Inhibit” and variations thereof refer to any measurable reduction of cellular activity. For example, inhibition of a particular cytokine refers to a decrease in production of the cytokine. The extent of inhibition may be characterized as a percentage of a normal level of activity.

“IRM compound” refers generally to an immune response modifier compound that alters the level of one or more immune regulatory molecules, e.g., cytokines or co-stimulatory markers, when administered to an IRM-responsive cell. Representative IRM compounds include, for example, the small organic molecules, purine derivatives, small heterocyclic compounds, amide derivatives, and oligonucleotide sequences described above.

“IRM moiety” refers to that portion of an immunomodulatory composition that possesses immunomodulatory activity. The IRM moiety may be, or be derived from, an IRM compound, but may, alternatively, be or be derived from some other immunomodulatory material. In some cases, the term “IRM moiety” may refer to an uncoupled compound prior to coupling to, or after uncoupling from, a targeting moiety.

“Marker” and variations thereof refer to any substance on a cell surface that may be bound by a ligand in a manner that is specific to some degree. As used herein, a marker-ligand interaction explicitly excludes immunological affinity—i.e., antibody-antigen affinity binding. Thus, some substances on the cell surface may be considered a marker (i.e., it may be capable of non-immunological receptor-ligand binding) in one context and an antigen in another context (i.e., it may be the target of an antibody).

“Prophylactic” and variations thereof refer to a treatment that limits, to any extent, the development and/or appearance of a symptom or clinical sign of a condition.

“Selective” and variations thereof refer to having a differential or a non-general impact on biological activity. An agonist that selectively modulates biological activity through a particular TLR may be a TLR-selective agonist. TLR-selectivity may be described with respect to a particular TLR (e.g., TLR8-selective or TLR7-selective) or with respect to a particular combination of TLRs (e.g., TLR 7/9-selective).

“Sign” or “clinical sign” refers to an objective physical finding relating to a particular condition capable of being found by one other than the patient.

“Specific” and variations thereof refer to having a differential or a non-general affinity, to any degree, for a particular target.

“Symptom” refers to any subjective evidence of disease or of a patient's condition.

“Targeting moiety” refers to that portion of an immunomodulatory composition that possesses target-specific affinity. The targeting moiety may be, or be derived from, an antibody, but may, alternatively, be or be derived from a non-antibody protein or peptide, or non-protein material including, for example, small molecules and/or nanoparticles. In some cases, the term “targeting moiety” may refer to an uncoupled compound prior to coupling to, or after uncoupling from, an IRM moiety.

“Therapeutic” and variations thereof refer to a treatment that ameliorates one or more existing symptoms or clinical signs associated with a condition.

“Treat” or variations thereof refer to reducing, limiting progression, ameliorating, preventing, or resolving, to any extent, the symptoms or signs related to a condition.

As used herein, “a,” “an,” “the,” “at least one,” and “one or more” are used interchangeably. Thus, for example, an immunomodulatory composition comprising “an” IRM compound can be interpreted to mean that the composition includes at least one IRM compound.

Also herein, the recitations of numerical ranges by endpoints include all numbers subsumed within that range (e.g., 1 to 5 includes 1, 1.5, 2, 2.75, 3, 3.80, 4, 5, etc.).

Many IRM compounds have been shown to stimulate certain aspects of the immune system. Often, this may occur through activation of dendritic cells (DCs), which are potent antigen presenting cells. Topical application of certain IRM compounds have been shown to be effective in several animal disease models and in human clinical trials (e.g., genital warts, actinic keratosis, superficial basal cell carcinoma, etc.). However, some IRM compounds also can induce undesirable side effects in some patients when administered systemically. This may be so, in part, because systemically administered IRM compounds may activate immune cells that are irrelevant to ameliorating a particular condition. For example, the activation of B cells may be irrelevant to treating certain forms of cancer. Treatment of such cancers with an IRM that is administered systemically may cause activation of B cells, which could lead to such side effects as, for example, non-specific immunoglobulin production, certain chronic condition (such as, for example, auto-immunity), and/or B cell depletion (which could lead to greater susceptibility of disease upon subsequent exposure to a pathogen).

Thus, in one aspect, the present invention provides immunomodulatory compositions that include an IRM moiety coupled to a targeting moiety. In some cases, the IRM moiety may be, or be derived from, an IRM compound. In some cases, the targeting moiety may be an antibody or be derived from an antibody (i.e., at least enough of the immunospecific portion of an antibody—e.g., enough of a light chain—to provide some degree of immunospecificity. However, in other cases, the targeting moiety may be, or be derived from, an agent that recognizes at least a portion of a tumor-specific marker such as, for example, a ligand that binds to a receptor that is, to some extent, specifically expressed by the target cell population. In this example, the receptor may be considered a tumor-specific marker.

The IRM moiety and the targeting moiety may be coupled directly or indirectly. For example, direct coupling of the IRM moiety and the targeting moiety may be accomplished through a covalent bond between the IRM moiety and the targeting moiety. Direct coupling also can be accomplished noncovalently by, for example, avidin-biotin affinity. One moiety may be biotinylated and the other moiety may be modified to contain an avidin moiety use known methods. The moieties so modified may be directly coupled by exploiting avidin-biotin affinity

Alternatively, the IRM moiety and the targeting moiety may be coupled indirectly by coupling each moiety to an intervening component such as, for example, a solid support or a spacer arm. Examples of, and methods for attaching IRM compounds to, suitable solid supports are described, for example, in U.S. Patent Publication No. US2004/0258698.

An intervening component (e.g., a solid support) may include a plurality of functional groups, thereby permitting the indirect coupling of a plurality of IRM moieties and/or a plurality of targeting moieties. When a plurality of IRM moieties is attached to an intervening component, the IRM moieties may be derived from the same IRM compound or from different IRM compounds. Likewise, when a plurality of targeting moieties is attached to an intervening component, the targeting moieties may be the same or different, offering the opportunity to design a composition having a high number of a particular targeting moiety (e.g., to increase the likelihood of finding a target) and/or the ability to bind to multiple targets (e.g., by having a number of different targeting moieties).

In some embodiments, an IRM moiety may be coupled to an anti-tumor targeting moiety such as, for example, a ligand of a tumor-specific marker or an anti-tumor antibody. As used herein, an anti-tumor antibody refers to an antibody (Ab) that recognizes cells of a tumor with some degree of specificity over normal tissue cells. The coupled IRM-Ab composition exploits the tumor specificity provided by the antibody to target delivery of the coupled IRM moiety to the vicinity of tumor antigens. Thus, dendritic cells in the vicinity of the tumor—as opposed to dendritic cells throughout the patient—are preferentially activated, thereby generating a localized tumor-specific, DC-mediated immune response while limiting systemic activation of dendritic cells that can induce general DC-mediated side effects. Therapy employing a coupled IRM/tumor-specific composition may be particularly desirable for treatment of cancers (e.g., metastatic cancers) that are difficult or impossible to treat by other therapies such as, for example, surgery, radiotherapy, etc.

Other examples of tumor-specific targeting moieties include certain non-protein materials such as, for example, nanoparticles and certain small molecules.

Nanoparticles that are about 1 nm to 200 nm in diameter may be used to provide tumor-specific delivery of IRM moieties to tumors. As noted above, an IRM compound may be attached to a nanoparticle by any suitable means such as, for example, covalent and noncovalent chemical interactions. Noncovalent chemical interactions can include affinity (e.g., avidin/biotin, antigen/antibody, receptor/ligand), ionic interaction, and/or hydrophobic interaction. Methods for attaching IRM compounds to solid supports such as nanoparticles are described, for example, in U.S. Patent Publication No. US2004/0258698.

Nanoparticles can possess tumor-specific targeting activity in at least two ways. First, as described above, the nanoparticle may be coated with a targeting moiety that directs the nanoparticle to a tumor. Methods for attaching targeting moieties (e.g., antibodies, receptor ligands, etc.) are well known. Second, nanoparticles may provide tumor-specific targeted delivery of an IRM moiety even without a having one or more targeting moieties attached. Nanoparticles having a diameter of from about 50 nm to about 200 nm may be delivered systemically and reside in bloodstream until they reach tumor vasculature. Localized changes in the porosity or permeability of the circulatory system permit the nanoparticles to escape the bloodstream, leave the circulatory system, and be deposited in the vicinity of the tumor.

One example of a small molecule moiety that can provide tumor-specific targeted delivery of an IRM moiety is bis-phosphonate. Bis-phosphonate functionality imparts high affinity, long-term association to the hydroxyapetite components of bone. Bis-phosphonates are known to be useful for targeted delivery and sequestering of diagnostic and/or therapeutic agents in bone. For example, bis-phosphonate drugs are used diagnostically for the delivery of bone imaging agents and therapeutically in osteoporosis, tumor osteolysis, and bone metastasis. An IRM/bis-phosphonate immunomodulatory composition could provide a depot of IRM within a common site of metastasis.

Leuteinizing hormone releasing hormone (LHRH) receptors are significantly elevated on breast cancer, prostate cancer, endometrial cancer, ovarian cancer, and melanoma cells. Thus, ligands of LHRH receptors may be used as targeting moieties in immunomodulatory compositions to provide tumor-specific targeted delivery of the IRM moiety to a tumor site. In animal models for the human cancers noted above, LHRH-directed therapeutics selectively home to the affected tissues. Coupling an IRM to a ligand of the LHRH receptor (e.g., LHRH or a synthetic analog) can provide targeted delivery of the IRM to tumor cells of these cancers, thereby concentrating the IRM at the site of the tumor and increasing the therapeutic index over that observed with the IRM compound alone. In studies comparing free Dox to LHRH-coupled Dox, approximately 200 times more free Dox was required to demonstrate an antitumor activity equal to the LHRH conjugate. Suitable LHRH receptor ligands could include LHRH decapeptide, an analog with agonist or antagonist activity, or a small molecule receptor ligand.

LHRH receptor is known to be overexpressed on many tumor cells (e.g., breast, prostate, melanoma) compared to normal organ tissues. Thus, a single IRM-LHRH receptor ligand coupled composition could be used for treating more than one cancer.

LHRH receptor ligands may be coupled directly to an IRM moiety or may be attached to nanoparticles to which one or more IRM moieties are also attached. Nanoparticles bearing LHRH receptor ligands have been shown to target breast cancer cells, whether within the breast or within metastases to the lung. By comparison, nanoparticles with LHRH preferentially traffic to the liver of normal animals.

Folic acid receptor ligands also may be useful as targeting moieties that may be coupled to an IRM moiety and provide tumor-specific targeted delivery of the IRM. The expression of folic acid receptors is increased on the surface of many tumor cells. Once again, coupling a folic acid receptor ligand to an IRM moiety can result in selective accumulation of the IRM at a tumor site, reducing systemic availability of the IRM moiety, and increasing the therapeutic index of the IRM moiety. Suitable folic acid receptor ligands include folic acid, an analog with agonist or antagonist activity, or a small molecule receptor ligand.

In some alternative embodiments, an IRM moiety may be coupled to a dendritic cell targeting moiety. The targeting moiety may be an antibody (e.g., an anti-DC antibody) or a non-antibody ligand that recognizes a DC-specific marker.

Suitable DC-specific markers may include, for example, a co-stimulatory marker such as, for example, any member of the TNFR Superfamily (e.g., CD40), CD70, CD80, CD86, B7-CD, B7.1, B7.2, etc. An immunomodulatory composition that includes a targeting moiety that recognizes a co-stimulatory marker may be used to deliver two DC-activating stimuli (i.e., IRM moiety and co-stimulation) in a single chemical entity.

As used herein, an anti-DC antibody refers to an antibody that recognizes a dendritic cell antigen. A suitable dendritic cell targeting moiety may bind to any antigen that is differentially expressed, either qualitatively or quantitatively, by dendritic cells. Suitable dendritic cell targeting moieties may bind to such antigens as, for example, DEC205, BDCA-1, BDCA-2, BDCA-3, BDCA-4, DC-SIGN, L-SIGN, HLR-DR, CD11c, CD13, CD14, CD21, CD33, CD35, CD123, C-type lectins, integrins (e.g., α4, α6, α1β1), and/or any one of the Toll-like receptors (TLRs), etc.

Regardless of whether the targeting moiety recognized a DC-specific marker or antigen, coupling the IRM moiety to the targeting moiety can limit systemic availability of the IRM moiety, even when administered via a systemic delivery route. Moreover, the IRM moiety may be concentrated in the vicinity of dendritic cells, thereby maturing and activating dendritic cells more effectively. Dendritic cells activated at the site of a tumor—or even inside a tumor mass—may be able to utilize a tumor antigen present on the surface of the tumor cells to initiate an immune response against the tumor. This method could provide a generalized anti-tumor therapy without the need for tumor-specific antibodies.

In other alternative embodiments, an IRM moiety may be coupled to an anti-macrophage targeting moiety. Macrophages are often localized in the vicinity of tumor cells. Thus, again, systemic availability of the IRM moiety can be limited, and the IRM moiety may be concentrated in the vicinity of the target cells (i.e., macrophages), thereby activating macrophages more efficiently. Activated macrophages are known to possess anti-tumor activity. Thus, this method could provide a generalized tumor therapy without the need for tumor-specific antibodies.

In other alternative embodiments, an IRM moiety may be coupled to a target specific moiety that recognizes a surface antigen on a cell type that can directly kill tumor cells such as, for example, CD8⁺ cytotoxic T cells, NK cells, or NKT cells. Once again, even if the immunomodulatory composition is administered systemically, the IRM moiety may be concentrated in the vicinity of the tumor-killing cells, thereby (a) activating tumor-killing cells more effectively, and/or (b) limiting the systemic availability of the IRM moiety. Tumor-killing cells activated at the site of a tumor—or even inside a tumor mass—may be able to utilize a tumor antigen present on the surface of the tumor cells to initiate an immune response against the tumor. This method could provide a generalized tumor therapy without the need for tumor-specific antibodies.

In other alternative embodiments, the IRM moiety may be coupled to a targeting moiety that recognizes, for example, an endothelial target. Significant differences exist in the endothelium environments of tumor masses compared to normal capillary beds. Differences exist, for example, in the identity and extent to which certain endothelial surface proteins, adhesion molecules (e.g., integrins), extracellular matrix proteins, growth factor receptors, etc. are expressed. These differences can be exploited to target delivery of an IRM moiety to tumor-related endothelium. Some reagents that specifically target such differences have been demonstrated to be useful as anti-angiogenic therapies. Coupling such an agent, as a targeting moiety, to an IRM moiety can combine two effective anti-tumor therapies: immunotherapy and anti-angiogenesis therapy.

Suitable anti-angiogenesis reagents include, for example, anti-CD105 antibodies (CD105 is overexpressed in tumor endothelium), anti-ED-B antibodies (ED-B is a fibronectin isoform found in tumor masses), peptides recognized by endothelial integrins associated with tumors, and growth factors whose receptors are upregulated on tumor endothelium (e.g., vascular endothelial growth factor).

The use of anti-angiogenic reagents in this way may offer the promise of combined anti-angiogenesis and immunotherapy. Additionally, targeted delivery of an IRM to the tumor endothelium, as opposed to the tumor itself, may provide more effective long-term treatment since, generally, the endothelium is a less mutagenic tissue than a tumor mass. Therefore, therapy directed toward the endothelium may be far less likely to cause drug resistance. Also, a therapy directed toward the endothelium may be effective against virtually any vascularized tumor (e.g., breast cancer, prostate cancer, lung cancer) without the need for tumor-specific reagents.

In still other alternative embodiments, the targeting moiety may include two or more targeting moieties, each of which could bind to a different target. Thus, for example, a targeting moiety may include one targeting moiety that recognizes, for example, an immune cell antigen or co-stimulatory marker (e.g., a dendritic cell target) and a second targeting moiety (e.g., an anti-tumor antigen) that recognizes, for example, target tumor cells. Such a composition may not only target delivery of the IRM moiety to either or both target cell populations, but also may provide targeted delivery of the target immune cell (e.g., dendritic cell or tumor-killing cell) and IRM moiety to the vicinity of the target tumor cells (e.g., a tumor).

The targeting moiety of the composition may be any material that can provide targeted delivery of the composition. In many embodiments, the targeting portion may provide immunospecific targeting, i.e., may be a sufficient portion of an immunoglobulin (i.e., an antibody) to promote immunospecific binding of the composition to a target antigen. However, the invention may be practiced using non-immunoglobulin targeting materials as well such as, for example, receptor ligands such as, for example, hormones (natural or synthetic), lipids, etc.

Because immunoglobulins are proteins, it is understood that modifications can be made to a particular immunoglobulin without rendering the modified immunoglobulin unsuitable for use as a targeting moiety. For example, one or more portions of the immunoglobulin amino acid sequence may be deleted or substituted, or additional amino acids may be added to an immunoglobulin, and the immunoglobulin can still retain sufficient immunospecific character to be suitable for practicing the invention. Therefore, in the description that follows, reference to a particular antibody includes modified immunoglobulins that have such modifications (e.g., amino acid additions, deletions, and/or substitutions) as are possible while retaining a sufficient amount of immunospecific character.

Suitable antibodies may be specific for microbial antigens (e.g., bacterial, viral, parasitic or fungal antigens), cancer or tumor-associated antigens, and/or self antigens. In many embodiments, a suitable antibody is one that recognizes and binds to an antigen present on or in a cell. An antibody that binds to a particular material (i.e., Antigen) may be referred to, interchangeably, as “anti-Antigen” or an “Antigen antibody”. In some instances, an antibody may be referred to by a generic name or commercial tradename.

Examples of suitable antibodies include, but are not limited to, RITUXAN (rituximab, anti-CD20 antibody), HERCEPTIN (trastuzumab), QUADRAMET, PANOREX, IDEC-Y2B8, BEC2, C225, ONCOLYM, SMART M195, ATRAGEN, OVAREX, BEXXAR, LDP-03, ior t6, MDX-210, MDX-11, MDX-22, OV103, 3622W94, anti-VEGF, ZENAPAX, MDX-220, MDX-447, MELIMMUNE-2, MELIMMUNE-1, CEACIDE [google], PRETARGET, NovoMAb-G2, TNT, Gliomab-H, GNI-250, EMD-72000, LYMPHOCIDE, CMA 676, Monopharm-C, 4B5, ior egf.r3, ior c5, BABS, anti-FLK-2, MDX-260, ANA Ab, SMART ID10Ab, SMART ABL 364 Ab, CC49 (mAb B72.3), ImmuRAIT-CEA, anti-IL-4 antibody, an anti-IL-5 antibody, an anti-IL-9 antibody, an anti-Ig antibody, an anti-IgE antibody, serum-derived hepatitis B antibodies, recombinant hepatitis B antibodies, anti-CD40 antibody, anti-OX40 antibody, anti-Cytokine Receptor antibodies, and the like.

Other antibodies similarly useful for the invention include alemtuzumab (B cell chronic lymphocytic leukemia), gemtuzumab ozogamicin (CD33+acute myeloid leukemia), hP67.6 (CD33+ acute myeloid leukemia), infliximab (inflammatory bowel disease and rheumatoid arthritis), ETANERCEPT (rheumatoid arthritis), tositumomab, MDX-210, oregovomab, anti-EGF receptor mAb, MDX-447, anti-tissue factor protein (TF), (Sunol); ior-c5, c5, edrecolomab, ibritumomab tiuxetan, anti-idiotypic mAb mimic of ganglioside GD3 epitope, anti-HLA-Dr10 mAb, anti-CD33 humanized mAb, anti-CD52 humAb, anti-CD1 mAb (ior t6), MDX-22, celogovab, anti-17-1A mAb, bevacizumab, daclizumab, anti-TAG-72 (MDX-220), anti-idiotypic mAb mimic of high molecular weight proteoglycan (1-MeI-1), anti-idiotypic mAb mimic of high molecular weight proteoglycan (1-MeI-2), anti-CEA Ab, hmAbH11, anti-DNA or DNA-associated proteins (histones) mAb, Gliomab-H mAb, GNI-250 mAb, anti-CD22, CMA 676), anti-idiotypic human mAb to GD2 ganglioside, ior egf/r3, anti-ior c2 glycoprotein mAb, ior c5, anti-FLK-2/FLT-3 mAb, anti-GD-2 bispecific mAb, antinuclear autoantibodies, anti-HLA-DR Ab, anti-CEA mAb, palivizumab, bevacizumab, alemtuzumab, BLyS-mAb, anti-VEGF2, anti-Trail receptor; B3 mAb, mAb BR96, breast cancer; and Abx-Cbl mAb.

Suitable antibodies also include the following:
Apoptosis antibodies such as, for example, Fas/Fas Ligand antibodies including, but not limited to, anti-human Fas/Fas Ligand antibodies, anti-murine Fas/Fas Ligand antibodies, Granzyme antibodies, Granzyme B antibodies; Bcl Antibodies including, but not limited to, anti-cytochrome C antibodies, anti-human Bcl antibodies (monoclonal), anti-human Bcl antibodies (polyclonal), anti-murine Bcl Antibodies (monoclonal), and anti-murine Bcl antibodies (polyclonal);
Miscellaneous apoptosis antibodies such as, for example, anti-TRADD, anti-TRAIL, and anti-DR3 antibodies;
Miscellaneous apoptosis related antibodies such as, for example, Bim antibodies including, but not limited to, anti-human, murine bim antibodies (polyclonal), anti-human, murine bim antibodies (monoclonal);
Caspase antibodies such as, for example, anti-human caspase antibodies (monoclonal), and anti-murine caspase antibodies;

Anti-CD antibodies such as, for example, anti-CD25, anti-CD29, anti-CD29, anti-CD41a, anti-CD42b, anti-CD42b, anti-CD42b, anti-CD43, anti-CD46, anti-CD61, anti-CD61, anti-CD62/P-slctn, anti-CD62/P-slctn, and anti-CD154;
Human chemokine antibodies such as, for example, human CNTF antibodies, human eotaxin antibodies, human epithelial neutrophil activating peptide-78 (ENA-78) antibodies, human exodus antibodies, human GRO antibodies, human HCC-1 antibodies, human I-309 antibodies, human IP-10 antibodies, human I-TAC antibodies, human LIF antibodies, human liver-expressed chemokine (LEC) antibodies, human lymphotaxin antibodies, human MCP antibodies, human MIP antibodies, human monokine induced by IFN-γ (MIG/CXCL9) antibodies, human NAP-2 antibodies, human NP-1 antibodies, human platelet factor-4 antibodies, human RANTES antibodies, human SDF antibodies, and human TECK antibodies;
Murine chemokine antibodies such as, for example, human B-cell attracting murine chemokine antibodies, chemokine-1 antibodies, murine eotaxin antibodies, murine exodus antibodies, murine GCP-2 antibodies, murine KC antibodies, murine MCP antibodies, murine MIP antibodies, and murine RANTES antibodies;
Rat Chemokine Antibodies such as, for example, rat CNTF antibodies, rat GRO antibodies, rat MCP antibodies, rat MIP antibodies, and rat RANTES antibodies;
Cytokine/cytokine receptor antibodies such as, for example, human biotinylated cytokine/cytokine receptor antibodies, human interferon (IFN) antibodies, human interleukin (IL) antibodies, human leptin antibodies, human oncostatin antibodies, human tumor necrosis factor (TNF) antibodies, human TNF receptor family antibodies, murine biotinylated cytokine/cytokine receptor antibodies, murine IFN antibodies, murine IL antibodies, murine TNF antibodies, murine TNF receptor antibodies, rat biotinylated cytokine/cytokine receptor antibodies, rat IFN antibodies, rat IL antibodies, and rat TNF antibodies;
Extracellular matrix antibodies such as, for example, collagen/procollagen antibodies, laminin antibodies, human collagen antibodies, human laminin antibodies, human procollagen antibodies, vitronectin/vitronectin receptor antibodies, hukman vitronectin antibodies, human vitronectin receptor antibodies, fibronectin/fibronectin receptor antibodies, human fibronectin antibodies, and human fibronectin receptor antibodies;
Growth factor antibodies such as, for example, human growth factor antibodies, murine growth factor antibodies, and porcine growth factor antibodies;
Miscellaneous antibodies such as, for example, baculovirus antibodies, cadherin antibodies, complement antibodies, Clq antibodies, Von Willebrand factor antibodies, Cre Antibodies, HIV Antibodies, influenza antibodies, human leptin antibodies, murine leptin antibodies, murine CTLA-4 antibodies, P450 antibodies, and RNA polymerase antibodies;
   Neurobiological antibodies such as, for example, amyloid antibodies, GFAP antibodies, human NGF antibodies, human NT-3 antibodies, and human NT-4 antibodies.

Additional antibodies suitable for use in the invention include, for example, antibodies listed in references such as the MSRS Catalog of Primary Antibodies and Linscott's Directory.

The immune response modifier moiety of the composition may be, or may be derived from, any suitable IRM compound. Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

In some embodiments, the IRM compound may be a small molecule immune response modifier (e.g., molecular weight of less than about 1000 Daltons). In some embodiments, the IRM compound may include a 2-aminopyridine fused to a five membered nitrogen-containing heterocyclic ring, or a 4-aminopyrimidine fused to a five membered nitrogen-containing heterocyclic ring.

IRM compounds suitable for use in the invention include compounds having a 2-aminopyridine fused to a five membered nitrogen-containing heterocyclic ring. Such compounds include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; and 1H-imidazo dimers fused to pyridine amines, quinoline amines, tetrahydroquinoline amines, naphthyridine amines, or tetrahydronaphthyridine amines.

In certain embodiments, the IRM compound may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In certain embodiments, the IRM compound may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a substituted imidazoquinoline amine refers to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amine, a 6-, 7-, 8-, or 9-aryl, heteroaryl, aryloxy or arylalkyleneoxy substituted imidazoquinoline amine, or an imidazoquinoline diamine. As used herein, substituted imidazoquinoline amines specifically and expressly exclude 1-(2-methylpropyl)-1H-imidazo[4,5-c]quinolin-4-amine and 4-amino-α,α-dimethyl-2-ethoxymethyl-1H-imidazo[4,5-c]quinolin-1-ethanol.

Suitable IRM compounds also may include the purine derivatives, imidazoquinoline amide derivatives, benzimidazole derivatives, adenine derivatives, aminoalkyl glucosaminide phosphates, and oligonucleotide sequences described above.

In one particular embodiment, the immune response modifier moiety of the compound is derived from N-[6-({2-[4-amino-2-(ethoxymethyl)-1H-imidazo[4,5-c]quinolin-1-yl]-1,1-dimethylethyl}amino)-6-oxohexyl]-4-azido-2-hydroxybenzamide.

In some embodiments of the present invention, the IRM compound may be an agonist of at least one TLR such as, for example, an agonist of TLR6, TLR7, or TLR8. The IRM may in some cases be an agonist of TLR9.

The TLR agonism for a particular compound may be assessed in any suitable manner. For example, assays and recombinant cell lines suitable for detecting TLR agonism of test compounds are described, for example, in U.S. Patent Publication Nos. US2004/0014779, US2004/0132079, US2004/0162309, US2004/0171086, US2004/0191833, and US2004/0197865.

Regardless of the particular assay employed, a compound can be identified as an agonist of a particular TLR if performing the assay with a compound results in at least a threshold increase of some biological activity mediated by the particular TLR. Conversely, a compound may be identified as not acting as an agonist of a specified TLR if, when used to perform an assay designed to detect biological activity mediated by the specified TLR, the compound fails to elicit a threshold increase in the biological activity. Unless otherwise indicated, an increase in biological activity refers to an increase in the same biological activity over that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine the TLR agonism of a compound in a particular assay.

The precise threshold increase of TLR-mediated biological activity for determining whether a particular compound is or is not an agonist of a particular TLR in a given assay may vary according to factors known in the art including but not limited to the biological activity observed as the endpoint of the assay, the method used to measure or detect the endpoint of the assay, the signal-to-noise ratio of the assay, the precision of the assay, and whether the same assay is being used to determine the agonism of a compound for both TLRs. Accordingly it is not practical to set forth generally the threshold increase of TLR-mediated biological activity required to identify a compound as being an agonist or a non-agonist of a particular TLR for all possible assays. Those of ordinary skill in the art, however, can readily determine the appropriate threshold with due consideration of such factors.

Assays employing HEK293 cells transfected with an expressible TLR structural gene may use a threshold of, for example, at least a three-fold increase in a TLR-mediated biological activity (e.g., NFκB activation) when the compound is provided at a concentration of, for example, from about 1 μM to about 10 μM for identifying a compound as an agonist of the TLR transfected into the cell. However, different thresholds and/or different concentration ranges may be suitable in certain circumstances. Also, different thresholds may be appropriate for different assays.

The immunomodulatory composition may be formulated in any manner suitable for administration to a subject. Suitable types of formulations are described, for example, in U.S. Pat. Nos. 5,736,553; 5,238,944; 5,939,090; 6,365,166; 6,245,776; and 6,486,186; European Patent No. EP 0 394 026; and U.S. Patent Publication No. US2003/0199538. The compound may be provided in any suitable form including but not limited to a solution, a suspension, an emulsion, or any form of mixture. The compound may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including but not limited to adjuvants, skin penetration enhancers, colorants, fragrances, flavorings, moisturizers, thickeners, and the like.

A formulation containing an immunomodulatory composition may be administered in any suitable manner such as, for example, non-parenterally or parenterally. As used herein, non-parenterally refers to administration through the digestive tract, including by oral ingestion. Parenterally refers to administration other than through the digestive tract such as, for example, intravenously, intramuscularly, transdermally, subcutaneously, transmucosally (e.g., by inhalation), or topically.

The composition of a formulation suitable for practicing the invention will vary according to factors known in the art including but not limited to the physical and chemical nature of the immunomodulatory composition, the nature of the carrier, the intended dosing regimen, the state of the subject's immune system (e.g., suppressed, compromised, stimulated), the method of administering the immunomodulatory composition, and the species to which the formulation is being administered. Accordingly, it is not practical to set forth generally the composition of a formulation effective for all possible applications. Those of ordinary skill in the art, however, can readily determine an appropriate formulation with due consideration of such factors.

In another aspect, the present invention includes a method of targeted delivery of an immune response modifier. Generally, the method includes administering to a subject an immunomodulatory composition that includes an immune response modifier coupled to a targeting moiety that recognizes a delivery target.

In another aspect, the present invention provides a method of inducing a localized immune response. Generally, the method includes administering to a subject an immunomodulatory composition that includes an immune response modifier coupled to a targeting moiety that recognizes a delivery target in an amount effective to induce an immune response.

In yet another aspect, the present invention provides a method of treating a condition in a subject treatable by inducing an immune response. Generally, the method includes administering to the subject an immunomodulatory composition that includes an immune response modifier coupled to a targeting moiety that recognizes a delivery target in an amount effective to treat at least one symptom or sign of the condition.

For each of the methods, suitable immunomodulatory compositions include the immunomodulatory composition described above. In some embodiments, the delivery target includes a tumor cell. In other embodiments, the delivery target includes an immune cell. In certain embodiments, the targeting moiety may recognize more than one delivery target. In one such case, one delivery target can include a tumor cell and a second delivery target can include an immune cell.

In some embodiments, the methods of the invention include administering the immunomodulatory composition to a subject in a formulation of, for example, from about 0.001% to about 10% (unless otherwise indicated, all percentages provided herein are weight/weight with respect to the total formulation) to the subject, although in some embodiments the immunomodulatory composition may be administered using a formulation that provides the immunomodulatory composition in a concentration outside of this range. In certain embodiments, the method includes administering to a subject a formulation that includes from about 0.01% to about 1% immunomodulatory composition, for example, a formulation that includes from about 0.1% to about 0.5% immunomodulatory composition.

An amount of an immunomodulatory composition effective for practicing the invention is an amount sufficient to generate a target-specific immune response. The precise amount of an immunomodulatory composition needed to practice the invention will vary according to factors known in the art including but not limited to the physical and chemical nature of the immunomodulatory composition, the nature of the carrier, the intended dosing regimen, the state of the subject's immune system (e.g., suppressed, compromised, stimulated), the method of administering the immunomodulatory composition, and the species to which the immunomodulatory composition is being administered. Accordingly, it is not practical to set forth generally the amount that constitutes an amount of immunomodulatory composition effective for all possible applications. Those of ordinary skill in the art, however, can readily determine the appropriate amount with due consideration of such factors.

In some embodiments, the methods of the present invention include administering sufficient immunomodulatory composition to provide a dose of the IRM moiety of, for example, from about 100 ng/kg to about 50 mg/kg to the subject, although in some embodiments the methods may be performed by administering immunomodulatory composition to provide a dose of the IRM moiety outside this range. In some of these embodiments, the method includes administering sufficient immunomodulatory composition to provide a dose of the IRM moiety of from about 10 μg/kg to about 5 mg/kg to the subject, for example, a dose of from about 100 μg/kg to about 1 mg/kg.

Alternatively, the dose may be calculated using actual body weight obtained just prior to the beginning of a treatment course. For the dosages calculated in this way, body surface area (m²) is calculated prior to the beginning of the treatment course using the Dubois method: m²=(wt kg^{0.425}×height cm^{0.725})×0.007184.

In some embodiments, the methods of the present invention may include administering sufficient IRM conjugate to provide a dose of, for example, from about 0.01 mg/m² to about 10 mg/m².

In some embodiments, the methods of the present invention include administering sufficient immunomodulatory composition to provide a dose of the targeting moiety of, for example, from about 50 ng/kg to about 100 mg/kg to the subject, although in some embodiments the methods may be performed by administering immunomodulatory composition to provide a dose of the targeting moiety outside this range. In some of these embodiments, the method includes administering sufficient immunomodulatory composition to provide a dose of the targeting moiety of from about 10 μg/kg to about 50 mg/kg to the subject, for example, a dose of from about 1 mg/kg to about 20 mg/kg.

The dosing regimen may depend at least in part on many factors known in the art including but not limited to the physical and chemical nature of the immunomodulatory composition, the nature of the carrier, the amount of immunomodulatory composition being administered, the state of the subject's immune system (e.g., suppressed, compromised, stimulated), the method of administering the immunomodulatory composition, and the species to which the immunomodulatory composition is being administered. Accordingly it is not practical to set forth generally the dosing regimen effective for all possible applications. Those of ordinary skill in the art, however, can readily determine an appropriate dosing regimen with due consideration of such factors.

In some embodiments of the invention, the immunomodulatory composition may be administered, for example, from a single dose to multiple doses. In certain embodiments, the immunomodulatory composition may be administered from about once per day to about once every three months, although in some embodiments the methods of the present invention may be performed by administering the immunomodulatory composition at a frequency outside this range. In one particular embodiment, the immunomodulatory composition is administered from about once per week to about once per month. In another embodiment, the immunomodulatory composition is administered once daily, two days per week. In yet another embodiment, the immunomodulatory composition is administered once daily three times per week.

Conditions that may be treated by administering an immunomodulatory composition include, but are not limited to:

(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picomavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

(c) other infectious diseases, such chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection;

(d) neoplastic diseases, such as solid tumor cancers (including, but not limited to breast cancer, colon cancer, pancreatic cancer, prostate cancer, lung cancer, prostate cancer, liver cancer, etc.), intraepithelial neoplasias, cervical dysplasia, actinic keratosis, basal cell carcinoma, squamous cell carcinoma, renal cell carcinoma, Kaposi's sarcoma, melanoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers;

(e) T_{H}2-mediated, atopic diseases, such as atopic dermatitis or eczema, eosinophilia, asthma, allergy, allergic rhinitis, and Ommen's syndrome;

(f) certain autoimmune diseases such as systemic lupus erythematosus, essential thrombocythaemia, multiple sclerosis, discoid lupus, alopecia greata; and

(g) diseases associated with wound repair such as, for example, inhibition of keloid formation and other types of scarring (e.g., enhancing wound healing, including chronic wounds).

Certain immunomodulatory compositions may be particularly helpful in individuals having compromised immune function. For example, certain immunomodulatory compositions may be used for treating the opportunistic infections and tumors that occur after suppression of cell mediated immunity in, for example, transplant patients, cancer patients and HIV patients.

The IRM moiety and the targeting moiety may be coupled by any suitable means including, for example, covalent and certain types of non-covalent coupling. Methods of covalently and non-covalently coupling an IRM compound and an antigen are described, for example, in U.S. Patent Publication No. US2004/0091491. Such methods, surprisingly, also may be used to covalently or non-covalently couple an IRM moiety and a targeting moiety so that each moiety of the resulting composition retains its functional character. It was believed that some of the conditions under which an IRM and antigen could be coupled (e.g., a pH of greater than 9.0 and UV irradiation) would destroy the target-specific character of certain targeting moieties (e.g., destroy the antigen recognition sites of antibody light chains). Moreover, steric considerations and the possibility that one or more IRM moieties would bind to and, therefore, block the target-binding portion of the targeting moiety were considered obstacles that would preclude using the methods of coupling an IRM and antigen for coupling an IRM and a targeting moiety.

Alternatively, a targeting moiety may be coupled to an IRM moiety using chemistry that does not depend upon UV irradiation to couple the IRM moiety and the targeting moiety. Such methods use chemistry that may make it easier to control the conjugation reaction, control the ratio of IRM moiety to targeting moiety, characterize the final composition, and obtain a more uniform product. Additional methods for coupling an IRM moiety and a targeting moiety are described, for example, in U.S. Provisional Patent Application entitled IMMUNE RESPONSE MODIFIER CONJUGATES, filed Feb. 22, 2006.

As noted above, an IRM moiety may be coupled to a targeting moiety using affinity interactions rather than covalent bonds. One example noted above exploits affinity between avidin and biotin. Alternative affinity interactions that may be useful for coupling an IRM moiety and a targeting moiety include, for example, glycoprotein/lectin interaction.

Alternatively, an immunomodulatory composition may be prepared by covalently coupling an IRM moiety and a targeting moiety. An immunomodulatory composition generally may be prepared by reacting an immune response modifier with a crosslinker and then reacting the resulting intermediate with a targeting moiety such as, for example, a sufficient portion of an antibody to provide the desired amount of target-specific delivery function. Many crosslinkers suitable for preparing bioconjugates are known and many are commercially available. See for example, Hermanson, G. (1996) Bioconjugate Techniques, Academic Press.

Alternatively, an immunomodulatory composition may be prepared, for example, according to the method shown in Reaction Scheme I in which a targeting moiety is linked to an IRM moiety through R₁ of the IRM moiety. In step (1) of Reaction Scheme I an IRM compound of Formula III is reacted with a heterobifunctional cross-linker of Formula IV to provide a compound of II. R_{A} and R_{B} each contain a functional group that is selected to react with the other. For example, if R_{A} contains a primary amine, then a heterobifunctional cross-linker may be selected in which R_{B} contains an amine-reactive functional group such as an N-hydroxysulfosuccinimidyl ester. R_{A} and R_{B} may be selected so that they react to provide the desired linker group in the conjugate.

Methods for preparing compounds of Formula III where R_{A} contains a functional group are known. See for example, U.S. Pat. Nos. 4,689,338; 4,929,624; U.S. Pat. Nos. 5,268,376; 5,389,640; 5,352,784; 5,494,916; 4,988,815; 5,367,076; 5,175,296; 5,395,937; 5,741,908; 5,693,811; 6,069,149; 6,194,425; 6,331,539; 6,451,810; 6,525,064;6,541,485; 6,545,016; 6,545,017; 6,656,938; 6,660,747; 6,664,260; 6,664,264; 6,670,372; 6,677,349; 6,683,088; and 6,797,718; and U.S. Patent Publication Nos. US2004/0147543 and US2004/0176367.

Many heterobifunctional cross-linkers are known and many are commercially available. See for example, Hermanson, G. (1996), Bioconjugate Techniques, Academic Press, Chapter 5 “Heterobifunctional Cross-Linkers”, 229-285. The reaction generally can be carried out by combining a solution of the compound of Formula III in a suitable solvent such as N,N-dimethylformamide with a solution of the heterobifunctional cross-linker of Formula IV in a suitable solvent such as N,N-dimethylformamide. The reaction may be run at ambient temperature. The product of Formula II may then be isolated using conventional techniques.

In step (2) of Reaction Scheme I a compound of Formula II that contains reactive group Z_{A} is reacted with the targeting moiety to provide the immunomodulatory conjugate of Formula I. The reaction generally can be carried out by combining a solution of the compound of Formula II in a suitable solvent such as dimethyl sulfoxide with a solution of the targeting moiety in a suitable buffer such as PBS. The reaction may be run at ambient temperature or at a reduced temperature (˜4° C.). If Z_{A} is a photoreactive group such as a phenyl azide then the reaction mixture will be exposed to long wave UV light for a length of time adequate to effect cross-linking (e.g., 10-20 minutes). The average number of IRM moieties per targeting moiety may be controlled by adjusting the amount of compound of Formula II used in the reaction. The immunomodulatory conjugate of Formula I may be isolated and purified using conventional techniques.

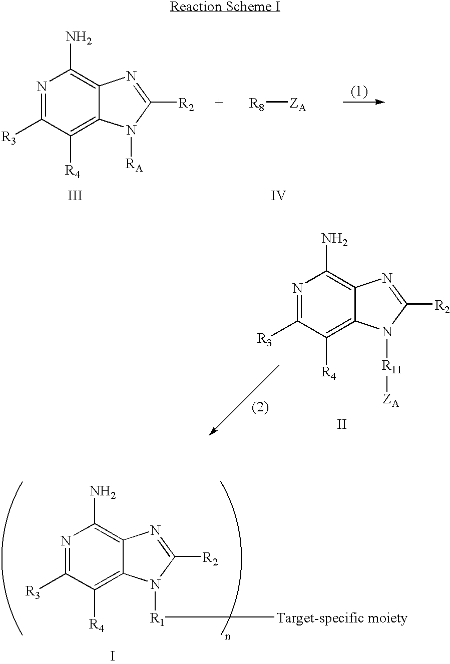

Alternatively, a compound of Formula II may be synthesized without using a heterobifunctional cross-linker. So long as the compound of Formula II contains the reactive group Z_{A}, it may be reacted with a targeting moiety using the method of step (2) above to provide an immunomodulatory conjugate.

As used herein, the terms “alkyl”, “alkenyl” and the prefix “alk-” include straight chain, branched chain, and cyclic groups, i.e. cycloalkyl and cycloalkenyl. Unless otherwise specified, these groups contain from 1 to 20 carbon atoms, with alkenyl groups containing from 2 to 20 carbon atoms. Preferred groups have a total of up to 10 carbon atoms. Cyclic groups can be monocyclic or polycyclic and preferably have from 3 to 10 ring carbon atoms. Exemplary cyclic groups include cyclopropyl, cyclopentyl, cyclohexyl, cyclopropylmethyl, and adamantyl.

The term “haloalkyl” is inclusive of groups that are substituted by one or more halogen atoms, including perfluorinated groups. This is also true of groups that include the prefix “halo-”. Examples of suitable haloalkyl groups are chloromethyl, trifluoromethyl, and the like.

The term “aryl” as used herein includes carbocyclic aromatic rings or ring systems. Examples of aryl groups include phenyl, naphthyl, biphenyl, fluorenyl and indenyl. The term “heteroaryl” includes aromatic rings or ring systems that contain at least one ring hetero atom (e.g., O, S, N). Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, and so on.

“Heterocyclyl” includes non-aromatic rings or ring systems that contain at least one ring hetero atom (e.g., O, S, N) and includes all of the fully saturated and partially unsaturated derivatives of the above mentioned heteroaryl groups. Exemplary heterocyclic groups include pyrrolidinyl, tetrahydrofuranyl, morpholinyl, thiomorpholinyl, piperidinyl, piperazinyl, thiazolidinyl, isothiazolidinyl, and imidazolidinyl.

The aryl, heteroaryl, and heterocyclyl groups can be unsubstituted or substituted by one or more substituents independently selected from the group consisting of alkyl, alkoxy, methylenedioxy, ethylenedioxy, alkylthio, haloalkyl, haloalkoxy, haloalkylthio, halogen, nitro, hydroxy, mercapto, cyano, carboxy, formyl, aryl, aryloxy, arylthio, arylalkoxy, arylalkylthio, heteroaryl, heteroaryloxy, heteroarylthio, heteroarylalkoxy, heteroarylalkylthio, amino, alkylamino, dialkylamino, heterocyclyl, heterocycloalkyl, alkylcarbonyl, alkenylcarbonyl, alkoxycarbonyl, haloalkylcarbonyl, haloalkoxycarbonyl, alkylthiocarbonyl, arylcarbonyl, heteroarylcarbonyl, aryloxycarbonyl, heteroaryloxycarbonyl, arylthiocarbonyl, heteroarylthiocarbonyl, alkanoyloxy, alkanoylthio, alkanoylamino, arylcarbonyloxy, arylcarbonythio, alkylaminosulfonyl, alkylsulfonyl, arylsulfonyl, heteroarylsulfonyl, aryldiazinyl, alkylsulfonylamino, arylsulfonylamino, arylalkylsulfonylamino, alkylcarbonylamino, alkenylcarbonylamino, arylcarbonylamino, arylalkylcarbonylamino, heteroarylcarbonylamino, heteroarylalkycarbonylamino, alkylsulfonylamino, alkenylsulfonylamino, arylsulfonylamino, arylalkylsulfonylamino, heteroarylsulfonylamino, heteroarylalkylsulfonylamino, alkylaminocarbonylamino, alkenylaminocarbonylamino, arylaminocarbonylamino, arylalkylaminocarbonylamino, heteroarylaminocarbonylamino, heteroarylalkylaminocarbonylamino and, in the case of heterocyclyl, oxo. If other groups are described as being “substituted” or “optionally substituted”, then those groups can also be substituted by one or more of the above-enumerated substituents.

Certain substituents are generally preferred. For example, preferred R₂ groups include hydrogen, alkyl groups having 1 to 4 carbon atoms (i.e., methyl, ethyl, propyl, isopropyl, n-butyl, sec-butyl, isobutyl, tert-butyl, and cyclopropylmethyl), and alkoxyalkyl groups (e.g., methoxyethyl and ethoxymethyl). Preferably R₃ and R₄ are independently hydrogen or methyl or R₃ and R₄ join together to form a benzene ring, a pyridine ring, a 6-membered saturated ring or a 6-membered saturated ring containing a nitrogen atom. One or more of these preferred substituents, if present, can be present in any combination.

Regardless of whether the IRM moiety and the targeting moiety are coupled covalently or noncovalently, an immunomodulatory composition may include an intervening component such as, for example, a spacer arm or a solid support. Certain spacer arms such as, for example, those having a length of from about 20 Å to about 100 Å, may improve solubility of the composition, thereby increasing the level of IRM activity obtainable using the composition. Suitable spacers are commercially available

In some embodiments, an immunomodulatory composition may include a macromolecular support to which both the targeting moiety and the IRM moiety are attached. In certain embodiments, the macromolecular support may be a solid support. The IRM moiety, targeting moiety, or both may be covalently attached to the macromolecular support using a linking group such as those described above. The macromolecular support may include, for example, supports such as those described in United States Patent Publication Nos. US2004/0202720 and US2004/0258698 such as, for example, agarose beads, gold particles, etc.

The methods of the present invention may be performed on any suitable subject. Suitable subjects include but are not limited to animals such as but not limited to humans, non-human primates, rodents, dogs, cats, horses, pigs, sheep, goats, or cows.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

In the examples below normal high performance flash chromatography (HPFC) was carried out using a HORIZON HPFC system (an automated high-performance flash purification product available from Biotage, Inc, Charlottesville, Va., USA) or an INTELLIFLASH Flash Chromatography System (an automated flash purification system available from AnaLogix, Inc, Burlington, Wis., USA). The eluent used for each purification is given in the example. In some chromatographic separations, the solvent mixture 80/18/2 v/v/v chloroform/methanol/concentrated ammonium hydroxide (CMA) was used as the polar component of the eluent. In these separations, CMA was mixed with chloroform in the indicated ratio.

### Preparation of the IRM Compounds

### IRM Compound 1 (IRM1): N-{2-[4-Amino-2-(ethoxymethyl)-1H-imidazo[4,5-c]quinolin-1-yl]-1,1-dimethylethyl}-6-[(3-mercaptopropanoyl)amino]hexanamide

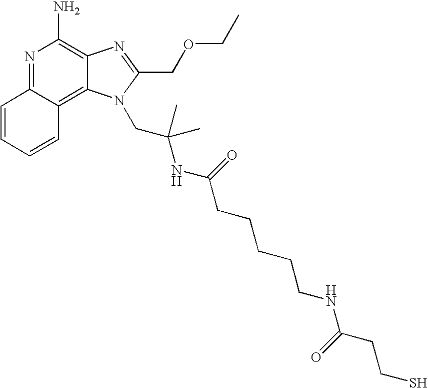 Part A

To a solution of 6-(carbobenzyloxyamino) caproic acid (8.49 grams (g), 32.0 millimole (mmol)) in DMF (50 mL) at 0° C. was added N-hydroxysuccinimide and 1-(3-dimethylaminopropyl)-3-ethylcarbodiimide hydrochloride (EDC). After a short period, the solution was added to a 0° C. solution of 1-(2-amino-2-methylpropyl)-2-(ethoxymethyl)-1H-imidazo[4,5-c]quinolin-4-amine (prepared as described in U.S. Patent Publication No. US2004/0091491, 10 g, 32 mmol) in DMF (100 mL). The mixture was allowed to warm to room temperature and was stirred for 3 days. The solution was diluted with water (400 mL) and extracted with ethyl acetate (3×). The organic layers were combined and washed with water (2×) and brine. The organic layer was dried over sodium sulfate, filtered, and concentrated. The crude product was purified by HPFC on silica gel three times (gradient elution with CMA in chloroform) to provide 4.90 g of benzyl 6-({2-[4-amino-2-(ethoxymethyl)-1H-imidazo[4,5-c]quinolin-1-yl]-1,1-dimethylethyl}amino)-6-oxohexylcarbamate as a white foam.

Part B

A mixture of benzyl 6-({2-[4-amino-2-(ethoxymethyl)-1H-imidazo[4,5-c]quinolin-1-yl]-1,1-dimethylethyl}amino)-6-oxohexylcarbamate (4.90 g, 8.75 mmol) and 10% palladium on carbon (0.5 g) in ethanol (100 mL) was hydrogenated on a Parr apparatus at 20-40 psi (1.4×10⁵-2.8×10⁵ Pa) for 1 day, during which time fresh hydrogen was introduced several times. The mixture was filtered through CELITE filter agent. The filtrate was concentrated under reduced pressure to yield a white foam that was used directly in the next step.

Part C

To a mixture of 3,3′-dithiodipropionic acid (920 mg, 4.38 mmol) and 1-hydroxybenzotriazole (HOBT) (1.42 g, 10.5 mmol) in dimethylformamide (DMF) (50 mL) at 0° C. was added 1-(3-dimethylaminopropyl)-3-ethylcarbodiimide hydrochloride (1.85 g, 9.63 mmol). The mixture was stirred at 0° C. for 4 hours. The material from Part B (8.75 mmol) was dissolved in DMF (20 mL), cooled to 0° C., and the cold solution of the activated diacid was added in one portion, with two DMF rinses (10 mL each). The reaction was allowed to warm slowly to room temperature overnight. Several more portions of EDC were added to the reaction at 0° C. over the next two days. The reaction was allowed to stir at room temperature for several days more, then was diluted with water and saturated aqueous sodium bicarbonate and was extracted with ethyl acetate several times. The combined organic extracts were washed with water and brine, and were concentrated under reduced pressure. The crude product was purified by HPFC to give 3.8 g of the disulfide dimer of N-{2-[4-amino-2-(ethoxymethyl)-1H-imidazo[4,5-c]quinolin-1-y]-1,1-dimethylethyl}-6-[(3-mercaptopropanoyl)amino]hexanamide.

Part D

The material from Part C (3.8 g, 3.7 mmol) was dissolved in methanol (30 mL) at room temperature. Tris(2-carboxyethyl)phosphine hydrochloride (1.38 g, 4.81 mmol) was added, followed by water (3 mL), and 12.5 M aqueous sodium hydroxide (1.12 mL, 14.1 mmol). The solution was stirred at room temperature for 2 hours and then was cooled to 0° C. The solution was adjusted to pH 6 with 1 M aqueous hydrochloric acid (approximately 14 mL). The methanol was removed under reduced pressure and aqueous sodium bicarbonate was added. The mixture was extracted with dichloromethane (3×). The organic extracts were combined, washed with water and brine, dried over sodium sulfate, filtered, and concentrated under reduced pressure. The crude product was purified by HPFC on silica gel (gradient elution with 0-50% CMA in chloroform). The appropriate fractions were concentrated to provide 2.36 g of N-{2-[4-amino-2-(ethoxymethyl)-1H-imidazo[4,5-c]quinolin-1-yl]-1,1-dimethylethyl}-6-[(3-mercaptopropanoyl)amino]hexanamide.

White foam, MS (ESI) m/z 515 (M+H)⁺. ¹H NMR (300 MHz, CDCl₃) δ 8.22 (dd, 1H), 7.80 (dd, 1H), 7.51 (ddd, 1H), 7.32 (ddd, 1H), 5.96 (m, 1H), 5.59 (s, 1H), 5.51 (br s, 2H), 5.06 (s, 2H), 4.84 (br s, 2H), 3.62 (q, J=6.9 Hz, 2H), 3.25 (q, J=6.9 Hz, 2H), 2.81 (m, 2H), 2.50 (t, J=6.9 Hz, 2H), 1.98 (m, 2H), 1.61-1.18 (m, 13H), 1.24 (t, J=6.9 Hz, 3H). Anal. calcd for C₂₆H₃₈N₆O₃S.0.5H₂O: C, 59.63; H, 7.51; N, 16.05; S, 6.12. Found: C, 59.89; H, 7.66; N, 16.22; S, 6.25.

### IRM Compound 2 (IRM2): N-{2-[4-Amino-2-(ethoxymethyl)-1H-imidazo[4,5-c]quinolin-1-yl]-1,1-dimethylethyl}-6-{[3-(pyridin-2-yldithio)propanoyl]amino} hexanamide

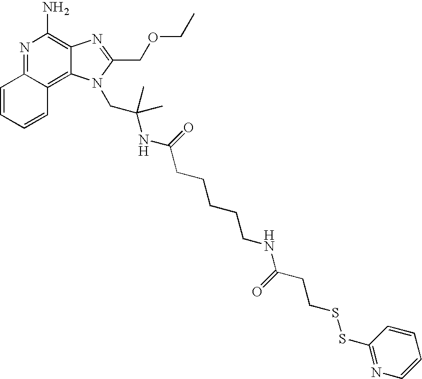

A solution of N-{2-[4-amino-2-(ethoxymethyl)-1H-imidazo[4,5-c]quinolin-1-yl]-1,1-dimethylethyl}-6-[(3-mercaptopropanoyl)amino]hexanamide (1.33 g, 2.58 mmol) in dichloromethane (16 mL) was added dropwise over 1.5 hours to a solution of 2,2′-dipyridyl disulfide (2.27 g, 10.3 mmol) in dichloromethane (10 mL). The solution was stirred at room temperature for 18 hours, then was concentrated under reduced pressure. The residue was purified by HPFC on silica gel twice (gradient elution with 1-10% methanol in dichloromethane) to yield 800 mg of N-{2-[4-amino-2-(ethoxymethyl)-1H-imidazo[4,5-c]quinolin-1-yl]-1,1-dimethylethyl}-6-{[3-(pyridin-2-yldithio)propanoyl]amino}hexanamide as a white foam.

Alternatively, N-{2-[4-amino-2-(ethoxymethyl)-1H-imidazo[4,5-c]quinolin-1-yl]-1,1-dimethylethyl}-6-{[3-(pyridin-2-yldithio)propanoyl]amino}hexanamide was synthesized from 6-amino-N-{2-[4-amino-2-(ethoxymethyl)-1H-imidazo[4,5-c]quinolin-1-yl]-1,1-dimethylethyl}hexanamide hydrochloride in one step. A mixture of 6-amino-N-{2-[4-amino-2-(ethoxymethyl)-1H-imidazo[4,5-c]quinolin-1-yl]-1,1-dimethylethyl}hexanamide hydrochloride (68 mg, 0.16 mmol), triethylamine (0.046 mL, 0.32 mmol), and N-succinimidyl 3-(2-pyridyldithio)propionate in tetrahydrofuran (1.6 mL) and DMF (0.5 mL) was stirred at room temperature for 5 hours. The reaction mixture was partitioned between water and ethyl acetate. The aqueous phase was extracted with ethyl acetate. The organic layers were combined, washed with water and brine, dried over sodium sulfate, filtered, and concentrated under reduced pressure. The crude product was purified by chromatography on silica gel to provide 36 mg of N-{2-[4-amino-2 -(ethoxymethyl)-1H-imidazo[4,5-c]quinolin-1-yl]-1,1-dimethylethyl}-6-{[3-(pyridin-2-yldithio)propanoyl]amino}hexanamide as a colorless oil.

MS (ESI) m/z 624 (M+H)⁺. ¹H NMR (300 MHz, CDCl₃) δ 8.43 (m, 1H), 8.23 (m, 1H), 7.80 (m, 1H), 7.63 (m, 2H), 7.50 (m, 1H), 7.32 (m, 1H), 7.11 sextet, J=4.4 Hz, 1H), 6.52 (m, 1H), 5.55 (s, 1H), 5.47 (br s, 2H), 5.07 (s, 2H), 4.83 (br s, 2H), 3.62 (q, J=6.9 Hz, 2H), 3.27 (q, 2H), 3.08 (t, J=6.9 Hz, 2H), 2.61 (t, 2H), 2.04-1.99 (m, 2H), 1.67-1.22 (m, 12H), 1.24 (t, J=7.0 Hz, 3H). Anal. calcd for C₃₁H₄₁N₇O₃S₂.1.0H₂O: C, 58.01; H, 6.75; N, 15.28; S, 9.99. Found: C, 58.37; H, 6.69; N, 15.24; S, 9.99.

### IRM Compound 3 (IRM3): N-[4-(4-Amino-2-butyl-1H-imidazo[4,5-c]quinolin-1-yl)butyl]-3-mercaptopropanamide

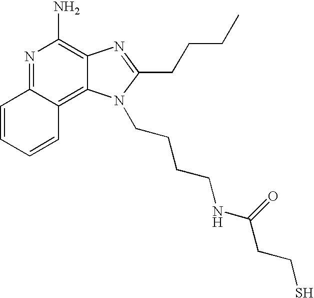 Part A

Following a procedure similar to that described above in Part C of IRM Compound 1,1-(4-aminobutyl)-2-butyl-1H-imidazo[4,5-c]quinolin-4-amine (U.S. Pat. No. 6,451,810 and references cited therein, 1.00 g, 3.21 mmol) was converted into 1.05 g of the disulfide dimer of N-[4-(4-amino-2-butyl-1H-imidazo[4,5-c]quinolin-1-yl)butyl]-3-mercaptopropanamide.

Part B

Following a procedure similar to that described above in Part D of IRM Compound 1, the disulfide dimer of N-[4-(4-amino-2-butyl-1H-imidazo[4,5-c]quinolin-1-yl)butyl]-3-mercaptopropanamide (0.78 g, 0.98 mmol) was converted into 600 mg of N-[4-(4-amino-2-butyl-1H-imidazo[4,5-c]quinolin-1-yl)butyl]-3-mercaptopropanamide after purification by HPFC on silica gel (gradient elution with 2-30% CMA in chloroform)

White solid, mp 133.0-135.0° C. MS (ESI) m/z 400 (M+H)⁺. ¹H NMR (300 MHz, CDCl₃) δ 7.90 (dd, J=8.1, 1.2 Hz, 1H), 7.82 (dd, J=8.1, 1.2 Hz, 1H), 7.50 (m, 1H), 7.32 (m, 1H), 5.50 (m, 1H), 5.34 (br s, 2H), 4.49 (t, J=7.5 Hz, 2H), 3.34 (q, J=6.8 Hz, 2H), 2.90 (m, 2H), 2.77 (q, J=6.9 Hz, 2H), 2.41 (t, J=6.8 Hz, 2H), 2.03-1.82 (m, 4H), 1.73-1.45 (m, 5H), 1.01 (t, J=7.5 Hz, 3H). Anal. calcd for C₂₁H₂₉N₅OS: C, 63.13; H, 7.32; N, 17.53. Found: C, 63.14; H, 7.36; N, 17.84.

### IRM Compound 4 (IRM4): N-[4-(4-Amino-2-butyl-1H-imidazo[4,5-c]quinolin-1-yl)butyl]-3-(pyridin-2-yldithio)propanamide

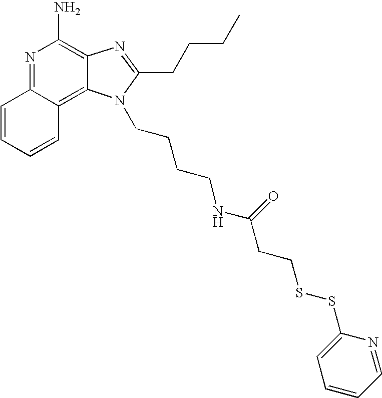 Part A

Following a procedure similar to that described above in Part A of IRM Compound 2, N-[4-(4-amino-2-butyl-1H-imidazo[4,5-c]quinolin-1-yl)butyl]-3-mercaptopropanamide (0.20 g, 0.50 mmol) was converted into 47 mg of N-[4-(4-amino-2-butyl-1H-imidazo[4,5-c]quinolin-1-yl)butyl]-3-(pyridin-2-yldithio)propanamide.

Yellow glassy solid, mp 63.0-73.0° C. MS (ESI) m/z 509 (M+H)⁺. ¹H NMR (300 MHz, CDCl₃) δ 8.33 (m, 1H), 7.89 (m, 1H), 7.81 (m, 1H), 7.61-7.46 (m, 3H), 7.30 (m, 1H), 7.05 (m, 1H), 6.77 (m, 1H), 5.49 (br s, 2H), 4.47 (t, J=7.5 Hz, 2H), 3.37 (q, J=6.5 Hz, 2H), 3.04 (m, 2H), 2.88 (m, 2H), 2.55 (t, J=6.5 Hz, 2H), 2.05-1.66 (m, 6H), 1.49 (sextet, J=7.5 Hz, 2H), 0.99 (t, J=7.5 Hz, 3H). Anal. calcd for C₂₆H₃₂N₆OS₂0.0.6H₂O: C, 60.11; H, 6.44; N, 16.18. Found: C, 59.80; H, 6.23; N, 16.25.

### IRM Compound 5 (IRM5): N-{2-[4-Amino-2-(ethoxymethyl)-1H-imidazo[4,5-c]quinolin-1-yl]-1,1-dimethylethyl}-4-hydrazino-4-oxobutanamide

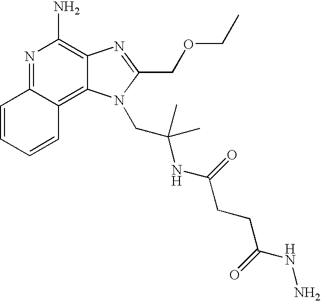 Part A

Succinic anhydride (3.20 g, 32.0 mmol) was added to a 100° C. solution of 1-(2-amino-2-methylpropyl)-2-(ethoxymethyl)-1H-imidazo[4,5-c]quinolin-4-amine (prepared as described in U.S. Patent Publication No. US2004/0091491, 2.00 g, 6.39 mmol) in DMF (20 mL). After 2 days, the reaction mixture was concentrated under reduced pressure to give an off-white solid. The solid was stirred with 100 mL of dichloromethane and was isolated by filtration. The filtrate was concentrated, stirred with dichloromethane (25 mL), and filtered to yield additional solid. The combined solids were dried under vacuum to give 3.16 g of 4-({2-[4-(2,5-dioxopyrrolidin-1-yl)-2-(ethoxymethyl)-1H-imidazo[4,5-c]quinolin-1-yl]-1,1-dimethylethyl}amino)-4-oxobutanoic acid as a white solid that was used without further purification.

Part B

A solution of 4-({2-[4-(2,5-dioxopyrrolidin-1-yl)-2-(ethoxymethyl)-1H-imidazo[4,5-c]quinolin-1-yl]-1,1-dimethylethyl}amino)-4-oxobutanoic acid (3.16 g, 6.39 mmol) in dichloromethane (100 mL) was treated with triethylamine (2.67 mL, 19.2 mmol), 1-(3-dimethylaminopropyl)-3-ethylcarbodiimide hydrochloride (3.67 g, 19.2 mmol), tert-butylcarbazate (2.46 g, 19.2 mmol) and N,N-dimethylpyridin-4-amine (78 mg, 0.64 mmol). The reaction mixture was stirred for 4 days and then was treated with 100 mL of water. The layers were separated and the aqueous portion was extracted with chloroform (50 mL). The combined organic layers were washed successively with water (50 mL) and brine (50 mL). The organic portion was dried over sodium sulfate, filtered, and concentrated under reduced pressure to give a white foam. The white foam was dissolved in dichloromethane (50 mL) and treated with ethylene diamine (1 mL). After stirring for 4 hours, the reaction mixture was treated with water (50 mL) and chloroform (50 mL) and the layers were separated. The aqueous portion was extracted with chloroform (2×25 mL). The combined organic layers were washed water (50 mL) and brine (50 mL). The organic layer was concentrated and purification of the crude product by chromatography on silica gel (gradient elution, 25%-100% CMA in chloroform) followed by crystallization from dichloromethane gave 1.67 g of tert-butyl 2-[4-({2-[4-amino-2-(ethoxymethyl)-1H-imidazo[4,5-c]quinolin-1-yl]-1,1-dimethylethyl}amino)-4-oxobutanoyl]hydrazinecarboxylate as a white powder.

Part C

A solution of tert-butyl 2-[4-({2-[4-amino-2-(ethoxymethyl)-1H-imidazo[4,5-c]quinolin-1-yl]-1,1-dimethylethyl}amino)-4-oxobutanoyl]hydrazinecarboxylate (792 mg, 1.50 mmol) in dichloromethane (30 mL) was treated with trifluoroacetic acid (3 mL). After stirring for 2 hours, an additional trifluoroacetic acid (3 mL) was added to the reaction mixture and stirring was continued for 1 hour. The reaction mixture was concentrated under reduced pressure and the resulting syrup was dissolved in water. The solution was made basic by the addition concentrated ammonium hydroxide and then was extracted repeatedly with 10% methanol/chloroform. The combined organic portions were and dried over sodium sulfate, filtered, and concentrated under reduced pressure. The crude white solid was purified by chromatography on silica gel (gradient elution, 25%-75% CMA in chloroform) gave 340 mg of N-{2-[4-amino-2-(ethoxymethyl)-1H-imidazo[4,5-c]quinolin-1-yl]-1,1-dimethylethyl}-4-hydrazino-4-oxobutanamide.

White solid, mp 203.0-204.6° C. ¹H NMR (300 MHz, DMSO-d₆) δ 8.98 (s, 1H), 8.32 (d, J=7.4 Hz, 1H), 7.74 (s, 1H), 7.60 (dd, J=8.3, 1.2, Hz, 1H), 7.41 (m, 1H), 7.23 (ddd, J=8.2, 7.0, 1.2 Hz, 1H), 6.59 (s, 2H), 4.99 (br s, 2H), 4.72 (br s, 2H), 4.16 (br s, 2H), 3.51 (q, J=7.0 Hz, 2H), 2.34-2.24 (m, 4H), 1.20 (br s, 6H), 1.13 (t, J=7.0 Hz, 3H); ¹³C NMR (125 MHz, DMSO-d₆) δ 172.3, 171.2, 152.4, 150.7, 145.8, 134.5, 127.0, 126.8, 126.7, 121.5, 121.0, 115.6, 65.8, 64.6, 55.1, 51.4, 31.8, 29.1, 25.9, 15.3; MS (ESI) m/z 428 (M+H)⁺; Anal. Calcd for C₂₁H₂₉N₇O₃0.0.5H₂O: C, 57.78; H, 6.93; N, 22.46. Found: C, 58.00; H, 6.69; N, 22.36.

### IRM Compound 6 (IRM6): N-{2-[4-Amino-2-(ethoxymethyl)-1H-imidazo[4,5-c]quinolin-1-yl]-1,1-dimethylethyl}-6-{[3-(2,5-dioxo-2,5-dihydro-1H-pyrrol-1-yl)propanoyl]amino}hexanamide

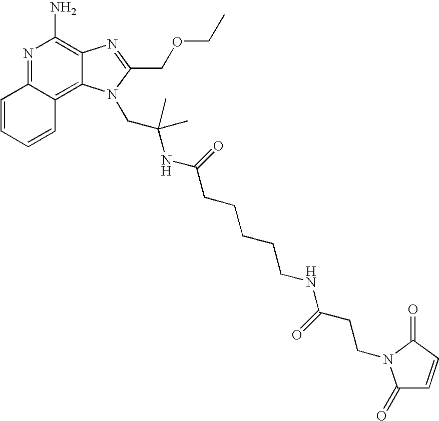 Part A

To a solution of 6-amino-N-{2-[4-amino-2-(ethoxymethyl)-1H-imidazo[4,5-c]quinolin-1-yl]-1,1-dimethylethyl}hexanamide (28 mg, 0.66 mmol, prepared as generally described in Part A-B of IRM Compound 1) in dichloromethane (0.5 mL) at room temperature was added N-succinimidyl-3-maleimidopropionate (18 mg, 0.68 mmol). The mixture was shaken until the reagent dissolved, allowed to stand for 30 minutes, then concentrated under reduced pressure. The foam was purified by reverse phase HPLC using 0.05% formic acid/acetonitrile in 0.05% formic acid/water as the eluent to yield 11 mg of N-{2-[4-amino-2-(ethoxymethyl)-1H-imidazo[4,5-c]quinolin-1-yl]-1,1-dimethylethyl}-6-{[3-(2,5-dioxo-2,5-dihydro-1H-pyrrol-1-yl)propanoyl]amino}hexanamide as the monoformate salt.

Off white glassy solid, MS (ESI) m/z 578 (M+H)⁺. ¹H NMR (300 MHz, DMSO-d₆) δ 8.34 (d, J=7.5 Hz, 1H), 8.22 (s, 2H), 7.90 (t, J=5.3 Hz, 1H), 7.65 (s, 1H), 7.60 (dd, J=8.4, 1.2 Hz, 1H), 7.42 (m, 1H), 7.22 (m, 1H), 7.00 (s, 2H), 6.67 (br s, 2H), 5.00 (br s, 2H), 4.74 (br, 2H), 3.59 (t, J=7.3 Hz, 2H), 3.51 (q, J=7.0 Hz, 2H), 2.98 (q, J=6.3 Hz, 2H), 2.31 (t, J=7.2 Hz, 2H), 2.04 (t, J=7.4 Hz, 2H), 1.47 (m, 2H), 1.36 (m, 2H), 1.20 (m, 8H), 1.13 (t, J=7.0 Hz, 3H).

### IRM Compound 7 (IRM7): N-{6-[(4-Amino-2-(ethoxymethyl)-1-{2-methyl-2-[(methylsulfonyl)amino]propyl}-1H-imidazo[4,5-c]quinolin-7-yl)oxy]hexyl}-4-azido-2-hydroxybenzamide

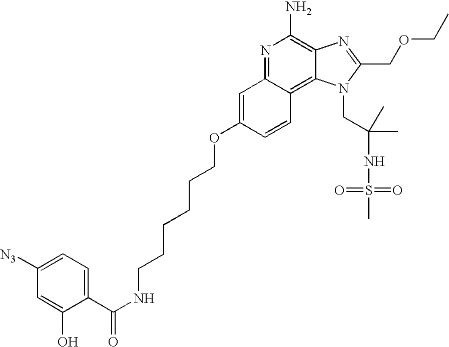

N-{2-[4-Amino-7-[(6-aminohexyl)oxy]-2-(ethoxymethyl)-1H-imidazo[4,5-c]quinolin-1-yl]-1,1-dimethylethyl}methanesulfonamide (prepared as described in Parts A-J of Example 45 in WO 2005/032484, 457 mg, 0.903 mmol) was dissolved in anhydrous DMF (9 mL) and treated with N-hydroxysuccinamidyl-4-azidosalacylic acid (248 mg, 0.903 mmol). The mixture was stirred under a nitrogen atmosphere overnight. The reaction mixture was then concentrated under reduced pressure. The resulting syrup was dissolved in dichloromethane (25 mL) and then washed with water (4×25 mL) and brine. The organic portion was dried over sodium sulfate, filtered, and concentrated under reduced pressure. Chromatography on silica gel (5% methanol/chloroform) gave a sticky, white solid that was concentrated from a mixture of dichloromethane and hexanes to give a white solid. The material was dried under vacuum at 50° C. for several days to give 380 mg of N-{6-[(4-amino-2-(ethoxymethyl)-1-{2-methyl-2-[(methylsulfonyl)amino]propyl}-1H-imidazo[4,5-c]quinolin-7-yl)oxy]hexyl}-4-azido-2-hydroxybenzamide as a white powder.

¹H NMR (300 MHz, DMSO-d₆) δ 8.54 (br s, 1H), 8.16 (d, J=9.1 Hz, 1H), 7.86 (d, J=8.5 Hz, 1H), 7.06 (d, J=2.6 Hz, 1H), 7.00 (s, 1H), 6.85 (dd, J=9.1, 2.6 Hz, 1H), 6.60 (dd, J=8.5, 2.3 Hz, 1H), 6.55 (d, J=2.1 Hz, 1H), 6.21 (s, 2H), 4.86 (s, 2H), 4.82 (s, 2H), 4.06 (t, J=6.4 Hz, 2H), 3.57 (q, J=7.0 Hz, 2H), 3.31 (q, J=6.5 Hz, 2H), 2.96 (s, 3H), 1.78 (m, 2H), 1.60 (m, 2H), 1.52-1.39 (m, 4H), 1.29 (s, 6H), 1.15 (t, J=7.0 Hz, 3H); ¹³C NMR (125 MHz, DMSO-d₆) δ 168.8, 162.1, 157.9, 152.5, 150.1, 147.3, 144.7, 135.0, 129.7, 125.3, 122.7, 112.6, 111.5, 110.0, 109.5, 108.2, 107.5, 67.6, 65.7, 65.1, 57.7, 54.6, 44.7, 39.3, 29.1, 29.0, 26.6, 25.8, 25.7, 15.3; MS m/z 668 (M+H)⁺; Anal. calcd for C₃₁H₄₁N₉O₆S: C, 55.76; H, 6.19; N, 18.88; S, 4.80. Found: C, 55.87; H, 6.28; N, 18.18; S, 4.66.

### IRM Compound 8 (IRM8): N-{6-[(4-Amino-1-{4-[(methylsulfonyl)amino]butyl}-2-propyl-1H-imidazo[4,5-c]quinolin-7-yl)oxy]hexyl}-4-azido-2-hydroxybenzamide

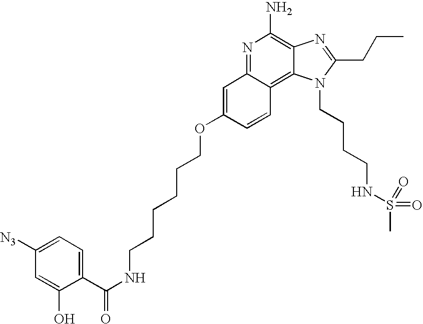

N-(4-{4-Amino-7-[(6-aminohexyl)oxy]-2-propyl-1H-imidazo[4,5-c]quinolin-1-yl}butyl)methanesulfonamide (prepared as described in Parts A-J of Example 47 in WO 2005/032484, 490 mg, 1.00 mmol) was dissolved in anhydrous DMF (10 mL) and treated with N-hydroxysuccinamidyl-4-azidosalacylic acid (274 mg, 1.00 mmol) and the mixture was stirred under nitrogen overnight. The reaction mixture was then concentrated under reduced pressure. The resulting syrup was dissolved in dichloromethane (25 mL) and then washed with water (3×25 mL) and brine. The organic portion was dried over sodium sulfate, filtered, and concentrated under reduced pressure. Chromatography on silica gel (5% methanol/chloroform) gave a sticky, white solid. The material was dried under vacuum at 50° C. for several days to give 400 mg of N-{6-[(4-amino-1-{4-[(methylsulfonyl)amino]butyl}-2-propyl-1H-imidazo[4,5-c]quinolin-7-yl)oxy]hexyl}-4-azido-2-hydroxybenzamide.

White powder. ¹H NMR (300 MHz, DMSO-d₆) δ 8.83 (t, J=5.3 Hz, 1H), 7.91 (d, J=9.0 Hz, 1H), 7.89 (d, J=8.6 Hz, 1H), 7.05 (d, J=2.6 Hz, 1H), 6.98 (t, J=5.8 Hz, 1H), 6.89 (dd, J=8.9, 2.6 Hz, 1H), 6.64 (dd, J=8.5, 2.3 Hz, 1H), 6.57 (d, J=2.3 Hz, 1H), 6.38 (s, 2H), 4.46 (t, J=7.2 Hz, 2H), 4.04 (t, J=6.4 Hz, 2H), 3.30 (q, J=6.5 Hz, 2H), 2.98 (q, J=6.4 Hz, 2H), 2.87 (t, J=7.4 Hz, 2H), 2.86 (s, 3H), 1.91-1.75 (m, 6H), 1.66-1.54 (m, 4H), 1.53-1.39 (m, 4H), 1.03 (t, J=7.4 Hz, 3H); ¹³C NMR (125 MHz, DMSO-d₆) δ 168.7, 162.1, 157.7, 152.5, 152.2, 146.7, 144.7, 133.1, 129.8, 125.3, 121.4, 112.7, 112.2, 109.9, 109.2, 108.3, 107.5, 44.7, 44.2, 39.5, 39.3, 29.1, 29.0, 28.7, 27.4, 26.7, 26.6, 25.7, 21.3, 14.2; MS m/z 652 (M+H)⁺; Anal. calcd for C₃₁H₄₁N₉O₅S: C, 57.13; H, 6.34; N, 19.34; S, 4.92. Found: C, 56.79; H, 6.05; N, 19.09; S, 4.79.

### IRM Compound 9 (IRM9): N-{6-[(4-Amino-2-propyl[1,3]thiazolo[4,5-c]quinolin-7-yl)oxy]hexyl}-4-azido-2-hydroxybenzamide

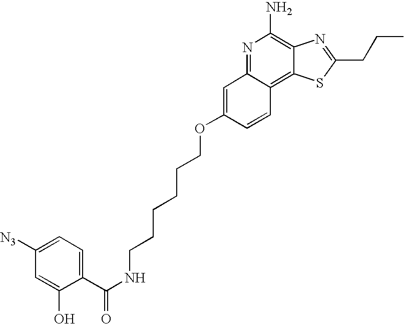

7-[(6-Aminohexyl)oxy]-2-propyl[1,3]thiazolo[4,5-c]quinolin-4-amine (prepared as described in Part A of Example 9 of U.S. Provisional Patent Application No. 60/733,036, 350 mg, 0.978 mmol) was dissolved in anhydrous DMF (10 mL) and treated with N-hydroxysuccinamidyl-4-azidosalacylic acid (268 mg, 0.978 mmol) and the mixture was stirred under a nitrogen atmosphere for 3 hours. The reaction mixture was then concentrated under reduced pressure. The resulting syrup was dissolved in dichloromethane (50 mL) and then washed with water (4×25 mL) and brine. The organic portion was dried over sodium sulfate, filtered, and concentrated under reduced pressure. Chromatography on silica gel (3% methanol/chloroform saturated with ammonium hydroxide) gave a sticky, light-yellow solid that still contained traces of DMF. The material was dissolved in dichloromethane (25 mL) and then washed with water (3×25 mL). The organic portion was dried over sodium sulfate, filtered, and concentrated under reduced pressure to give 330 mg of N-{6-[(4-amino-2-propyl[1,3]thiazolo[4,5-c]quinolin-7-yl)oxy]hexyl}-4-azido-2-hydroxybenzamide.

Light-yellow foam, mp 127-128° C. ¹H NMR (300 MHz, DMSO-d₆) δ 13.12 (s, 1H), 8.80 (t, J=5.3 Hz, 1H), 7.88 (d, J=8.6 Hz, 1H), 7.64 (d, J=8.7 Hz, 1H), 7.02 (d, J=2.4 Hz, 1H), 6.88 (dd, J=8.8, 2.5 Hz, 1H), 6.78 (s, 2H), 6.64 (dd, J=8.5, 2.3 Hz, 1H), 6.57 (d, J=2.3 Hz, 1H), 4.06 (t, J=6.4 Hz, 2H), 3.29 (t, J=6.9 Hz, 2H), 3.11 (t, J=7.5 Hz, 2H) 1.86 (m, 2H), 1.77 (m, 2H), 1.58 (m, 2H), 1.51-1.35 (m, 4H), 1.02 (t, J=7.3 Hz, 3H); ¹³C NMR (125 MHz, DMSO-d₆) δ 169.4, 168.8, 162.0, 159.8, 152.7, 146.9, 144.7, 139.8, 136.6, 129.8, 125.9, 113.8, 113.7, 113.1, 109.9, 108.4, 107.5, 68.2, 39.4, 35.6, 29.1, 29.0, 26.6, 25.6, 22.8, 13.6; MS m/z 520 (M+H)⁺; Anal. calcd for C₂₆H₂₉N₇O₃S: C, 60.10; H, 5.63; N, 18.87; S, 6.17. Found: C, 59.97; H, 5.34; N, 18.63; S, 6.21.

### Control Compound 1 (CC1): N-{2-[2-(Ethoxymethyl)-1H-imidazo[4,5-c]quinolin-1-yl]-1,1-dimethylethyl}-6-[(3-mercaptopropanoyl)amino]hexanamide

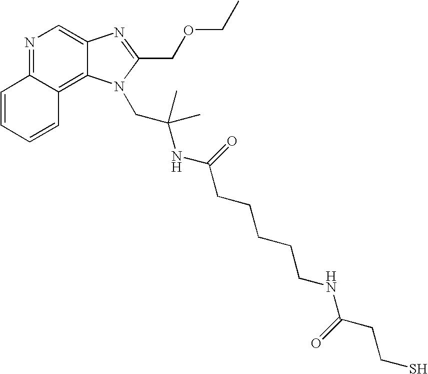 Part A

6-[(tert-Butoxycarbonyl)amino]hexanoic acid (6.81 g, 29.5 mmol), 1-hydroxybenzotriazole (3.98 g, 29.5 mmol), and 1-(3-dimethylaminopropyl)-3 -ethylcarbodiimide hydrochloride (EDC) (5.66 g, 29.5 mmol) were added to a 0° C. solution of 2-[2-(ethoxymethyl)-1H-imidazo[4,5-c]quinolin-1-yl]-1,1-dimethylethylamine (which was prepared by acid-mediated deprotection of tert-butyl 2-[2-(ethoxymethyl)-1H-imidazo[4,5-c]quinolin-1-yl]-1,1-dimethylethylcarbamate, which is described in U.S. Patent Publication No. US2004/0091491, 8.00 g, 26.8 mmol) in DMF (80 mL). The solution was stirred at room temperature overnight. More 6-[(tert-butoxycarbonyl)amino]hexanoic acid and EDC were added and the solution was stirred for an additional hour. The solution was partitioned between aqueous sodium bicarbonate and ethyl acetate. The aqueous layer was extracted with ethyl acetate (2×). The organic layers were combined, washed with water and brine, dried over sodium sulfate, filtered, and concentrated under reduced pressure. The crude product was purified by chromatography on silica gel to provide 3.99 g of tert-butyl 6-({2-[2-(ethoxymethyl)-1H-imidazo[4,5-c]quinolin-1-yl]-1,1-dimethylethyl}amino)-6-oxohexylcarbamate as a foam.

Part B

Trifluoroacetic acid (30 mL) was added slowly to a solution of tert-butyl 6-({2-[2-(ethoxymethyl)-1H-imidazo[4,5-c]quinolin-1-yl]-1,1-dimethylethyl}amino)-6-oxohexylcarbamate (3.99 g, 7.82 mmol) in dichloromethane (80 mL) at room temperature. After 1.5 hours, the solution was concentrated under pressure to afford an oil. The oil was dissolved in a small amount of water and concentrated ammonium hydroxide. The resulting basic mixture was extracted with dichloromethane multiple times. The combined organic extracts were dried over sodium sulfate, filtered, and concentrated under reduced pressure to afford 3.4 g of 6-amino-N-{2-[2-(ethoxymethyl)-1H-imidazo[4,5-c]quinolin-1-yl]-1,1-dimethylethyl}hexanamide, which was used in the next step without further purification.

Part C

A solution of 6-amino-N-{2-[2-(ethoxymethyl)-1H-imidazo[4,5-c]quinolin-1-yl]-1,1-dimethylethyl}hexanamide (1.35 g, 3.28 mmol), 3,3′-dithiodipropionic acid (0.345 g, 1.64 mmol), 1-hydroxybenzotriazole (0.443 g, 3.28 mmol), and 1-(3-dimethylaminopropyl)-3-ethylcarbodiimide hydrochloroide (0.692, 3.61 mmol) in DMF (10 mL) was stirred at room temperature overnight. The solution was concentrated under reduced pressure and partitioned between saturated aqueous sodium bicarbonate and ethyl acetate/methanol. The organic layers were combined, dried over sodium sulfate, filtered, and concentrated under reduced pressure. The crude product was purified by HPFC on silica gel (gradient elution with CMA/chloroform) to provide the disulfide dimer of N-{2-[2-(ethoxymethyl)-1H-imidazo[4,5-c]quinolin-1-yl]-1,1-dimethylethyl}-6-[(3-mercaptopropanoyl)amino]hexanamide.

Part D

N-{2-[2-(Ethoxymethyl)-1H-imidazo[4,5-c]quinolin-1-yl]-1,1-dimethylethyl}-6-[(3-mercaptopropanoyl)amino]hexanamide (2.14 g, 2.15 mmol) was dissolved in methanol (20 mL). Tris(2-carboxyethyl)phosphine (0.800 g, 2.79 mmol), water (2 mL), and 12.5 M NaOH (0.65 mL, 8.17 mmol) were added. The solution was allowed to stir for 1.5 hours at room temperature, then was cooled in an ice bath. The solution was adjusted to pH 6 with 1 M HCl and the resulting mixture was concentrated under reduced pressure to remove the methanol. The mixture was partitioned between saturated aqueous sodium bicarbonate and dichloromethane. The aqueous layer was extracted multiple times with dichloromethane. The organic phases were combined, washed with water and brine, dried over sodium sulfate, filtered, and concentrated under reduced pressure to provide 1.69 g of N-{2-[2-(ethoxymethyl)-1H-imidazo[4,5-c]quinolin-1-yl]-1,1-dimethylethyl}-6-[(3-mercaptopropanoyl)amino]hexanamide as a white foam that was heated under vacuum to produce a glassy solid.

Colorless glassy solid. MS (ESI) m/z 500 (M+H)⁺. ¹H NMR (300 MHz, CDCl₃) δ 9.30 (s, 1H), 8.52 (m, 1H), 8.26 (m, 1H), 7.72-7.62 (m, 2H), 5.84 (m, 1H), 5.59 (br s, 1H), 5.18 (br s, 2H), 4.91 (br s, 2H) 3.63 (m, 2H), 3.27 (m, 2H), 2.81 (m, 2H), 2.49 (t, J=6.9 Hz, 2H), 2.05 (t, J=7.5 Hz, 2H), 1.63-1.22 (m, 16H). Anal. calcd for C₂₆H₃₇N₅O₃S: C, 62.50; H, 7.46; N, 14.02; S, 6.42. Found: C, 62.23; H, 7.54; N, 13.90; S, 6.65.

### Control Compound 2 (CC2): N-{2-[2-(Ethoxymethyl)-1H-imidazo[4,5-c]quinolin-1-yl]-1,1-dimethylethyl}-6-{[3-(pyridin-2-yldithio)propanoyl]amino}hexanamide

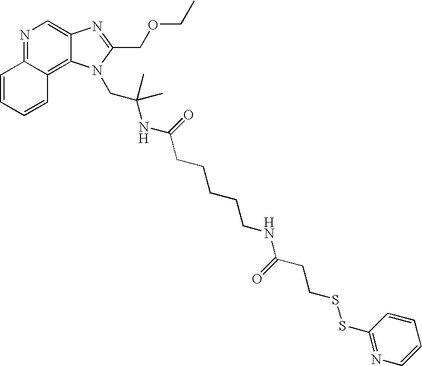

N-{2-[2-(Ethoxymethyl)-1H-imidazo[4,5-c]quinolin-1-yl]-1,1-dimethylethyl}-6-{[3-(pyridin-2-yldithio)propanoyl]amino}hexanamide (0.55 g) was prepared from N-{2-[2-(ethoxymethyl)-1H-imidazo[4,5-c]quinolin-1-yl]-1,1-dimethylethyl}-6-[(3-mercaptopropanoyl)amino]hexanamide (0.83 g) using the procedure described for the preparation of IRM Compound 2. The final product was purified by HPFC on silica gel (gradient elution with 1-10% methanol in dichloromethane) and was isolated as a colorless glassy solid after heating under vacuum.

Colorless glassy solid. MS (ESI) m/z 609 (M+H)⁺. ¹H NMR (300 MHz, CDCl₃) δ 9.30 (s, 1H), 8.53 (m, 1H), 8.43 (m, 1H), 8.26 (m, 1H), 7.71-7.60 (m, 4H), 7.11 (m, 1H), 6.50 (m, 1H), 5.58 (br s, 1H), 5.19 (br s, 2H), 4.90 (br s, 2H) 3.63 (q, J=6.9 Hz, 2H), 3.28 (m, 2H), 3.07 (m, 2H), 2.60 (m, 2H), 2.07 (t, J=7.5 Hz, 2H), 1.65-1.22 (m, 12H), 1.24 (t, J=6.9 Hz, 3H). Anal. calcd for C₃₁H₄₀N₆O₃S₃0.0.15CH₃OH: C, 60.97; H, 6.67; N, 13.70; S, 10.43. Found: C, 60.57; H, 6.75; N, 13.61; S, 10.62.

### Control Compound 3 (CC3): N-{2-[2-(Ethoxymethyl)-1H-imidazo[4,5-c]quinolin-1-yl]-1,1-dimethylethyl}-4-hydrazino-4-oxobutanamide

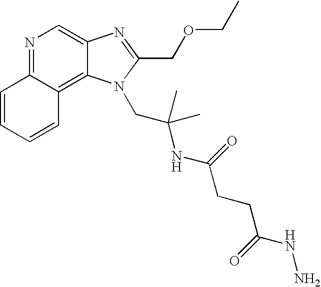 Part A

DMF (10 mL) was added to a mixture of 2-[2-(ethoxymethyl)-1H-imidazo[4,5-c]quinolin-1-yl]-1,1-dimethylethylamine (which was prepared by acid-mediated deprotection of tert-butyl 2-[2-(ethoxymethyl)-1H-imidazo[4,5-c]quinolin-1-yl]-1,1-dimethylethylcarbamate, which is described in U.S. Patent Publication No. US2004/0091491, 1.00 g, 3.36 mmol) and succinic anhydride (0.336 g, 3.36 mmol) at room temperature. The mixture was sonicated briefly until a solution formed. The solution was allowed to stand at room temperature for 3 days and then was used in the next step.

Part B

The solution from Part A was cooled to 0° C. and was tert-butyl carbazate (0.489 g, 3.70 mmol) and 1-(3-dimethylaminopropyl)-3-ethylcarbodiimide hydrochloride (0.709 g, 3.70 mmol) were added. The mixture was allowed to warm to room temperature and was stirred overnight. More tert-butyl carbazate and 1-(3-dimethylaminopropyl)-3-ethylcarbodiimide hydrochloride were added. After 1 hour, the solution was diluted with water (60 mL) and extracted with ethyl acetate (3×75 mL). The combined organic layers were washed with water, saturated aqueous sodium bicarbonate, and brine. The organic layer was dried over sodium sulfate, filtered, and concentrated under reduced pressure to yield a yellow foam. Chloroform was added to the foam causing a fine white solid to form. The solid was isolated by filtration to provide 0.758 g of tert-butyl 2-[4-({2-[2-(ethoxymethyl)-1H-imidazo[4,5-c]quinolin-1-yl]-1,1-dimethylethyl}amino)-4-oxobutanoyl]hydrazinecarboxylate.

Part C

Trifluoroacetic acid (3 mL) was added slowly to a solution of tert-butyl 2-[4-({2-[2-(ethoxymethyl)-1H-imidazo[4,5-c]quinolin-1-yl]-1,1-dimethylethyl}amino)-4-oxobutanoyl]hydrazinecarboxylate (0.688 g, 1.34 mmol) in dichloromethane (7 mL). The solution was stirred for 2.5 hours, then was concentrated under reduced pressure. The trifluoroacetic acid salt of N-{2-[2-(ethoxymethyl)-1H-imidazo[4,5-c]quinolin-1-yl]-1,1-dimethylethyl}-4-hydrazino-4-oxobutanamide was applied to anion exchange resins, which were eluted with pyridine in methanol to provide N-{2-[2-(ethoxymethyl)-1H-imidazo[4,5-c]quinolin-1-yl]-1,1-dimethylethyl}-4-hydrazino-4-oxobutanamide as a free base, which was purified by HPFC on silica gel (gradient elution, 2-50% CMA in chloroform). The appropriate fractions were concentrated under reduced pressure to yield a foam that was heated under vacuum to afford 0.31 g of N-{2-[2-(ethoxymethyl)-1H-imidazo[4,5-c]quinolin-1-yl]-1,1-dimethylethyl}-4-hydrazino-4-oxobutanamide as a glassy solid.

Glassy solid. MS (ESI) m/z 413 (M+H)⁺. ¹H NMR (300 MHz, CDCl₃) δ 9.30 (s, 1H), 8.50 (m, 1H), 8.26 (m, 1H), 7.71-7.61 (m, 2H), 7.14 (br s, 1H), 6.13 (br s, 1H), 5.16 (brs, 2H), 4.90 (br s, 2H), 3.88 (br s, 2H), 3.63 (q, J=6.9 Hz, 2H), 2.41 (s, 4H), 1.37 (br s, 6H), 1.24 (t, J=6.9 Hz, 3H). Anal. Calcd for C₂₁H₂₈N₆O₃0.0.4H₂O: C, 60.10; H, 6.92; N, 20.02. Found: C, 60.37; H, 7.01; N, 20.01.

Control Compound 4 (CC4): N-{2-[2-(Ethoxymethyl)-1H-imidazo[4,5-c]quinolin-1-yl]-1,1-dimethylethyl}-6-{[3-(2,5-dioxo-2,5-dihydro-1H-pyrrol-1-yl)propanoyl]amino} hexanamide

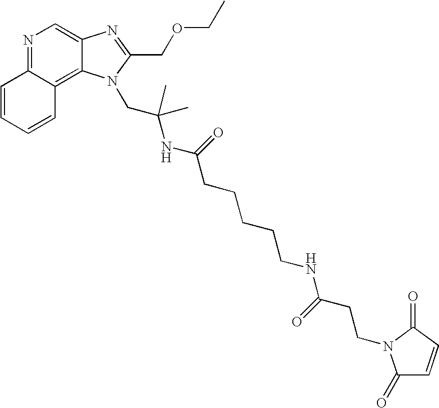

To a solution of 6-amino-N-{2-[2-(ethoxymethyl)-1H-imidazo[4,5-c]quinolin-1-yl]-1,1-dimethylethyl}hexanamide (which was prepared as described above in Parts A and B of Control Compound 1, 163 mg, 0.40 mmol) in dichloromethane (4 mL) at room temperature was added N-succinimidyl-3-maleimidopropionate (111 mg, 0.42 mmol). The mixture was shaken until the reagent dissolved and allowed to stand overnight. The mixture was diluted with dichloromethane (25 mL), washed with 2M aqueous ammonia (10 mL), dried over magnesium sulfate and concentrated under reduced pressure to provide 158 mg of N-{2-[2-(ethoxymethyl)-1H-imidazo[4,5-c]quinolin-1-yl]-1,1-dimethylethyl}-6-{[3-(2,5-dioxo-2,5-dihydro-1H-pyrrol-1-yl)propanoyl]amino}hexanamide.

Light yellow foam, MS (ESI) m/z 563 (M+H)⁺. ¹H NMR (300 MHz, CDCl₃) δ 9.30 (s, 1H), 8.55 (d, J=7.0 Hz, 1H), 8.31 (m, 1H), 7.69 (m, 2H), 6.68 (s, 2H), 5.79 (m, 1H), 5.57 (br s, 1H), 5.20 (m, 2H), 4.92 (m, 2H), 3.83 (t, J=7.1 Hz, 2H), 3.63 (q, J=6.9 Hz, 2H), 3.22 (q, J=6.7 Hz, 2H), 2.52 (t, J=7.2 Hz, 2H), 2.05 (t, J=7.3 Hz, 2H), 1.55 (m, 12H), 1.25 (t, J=7.0 Hz, 3H).

Additional The IRM compounds used in the examples are shown in Table 1.

TABLE 1

| Compound | Chemical Name | Reference |
|---|---|---|
| IRM10 | N-[6-({2-[4-amino-2-(ethoxymethyl)- | US 2004/0091491 |
| | 1H-imidazo[4,5-c]quinolin-1-yl]-1,1- | |
| | dimethylethyl}amino)-6-oxohexyl]-4- | |
| | azido-2-hydroxybenzamide | |

### Preparation of Modified Antibodies

Materials used to prepare the antibody-IRM conjugates can be found in Table 2 below.

TABLE 2

| Material | Source |
|---|---|
| Phosphate Buffered Saline (PBS), pH 7.4 | Biosource (Camarillo, CA) |
| Phosphate Buffered Saline (PBS), pH 7.2 | Biosource |
| 1 N Sodium Hydroxide (NaOH) | J. T. Baker (Phillipsburg, NJ) |
| MULTIWELL 12 Well Tissue Culture | Becton Dickenson |
| Plate | (Franklin Lakes, NJ) |
| Beckman SYSTMEM GOLD 126 Solvent | Beckman Coulter |
| Module/168 detector chromatography | (Fullerton, CA) |
| system | |
| SUPERDEX 200 10/300 GL Size | Amersham Biosciences/GE |
| Exclusion Column | Healthcare (Piscataway, NJ) |
| 4-succinimidyloxycarbonyl-α-methyl-α- | Pierce (Rockford, IL) |
| [2-pyridyldithio]toluene (SMPT) | |
| sulfosuccinimidyl 4-(N- | Pierce |
| maleimidomethyl)cyclohexane-1- | |
| carboxylate (Sulfo-SMCC) | |
| NHS-PEO₈-Maleimide (succinimidyl-[(N- | Pierce |
| maleimidopropionamido)- | |
| octaethyleneglycol] ester) | |
| Ellman's Reagent (5,5′-dithio-bis-(2- | Pierce |
| nitrobenzoic acid) | |
| Traut's Reagent (2-Iminothiolane.HCl; 2- | Pierce |
| IT) | |
| Dimethylsulfoxide (DMSO) | EMD (Gibbstown, NJ) |
| L-cysteine HCl | Pierce |
| 0.5M EDTA, pH 8.0 | Promega (San Luis |
| | Obispo, CA) |
| 1M Tris, pH 8.0 | Biosource |
| PD10 Desalting Column | Amersham Biosciences/ |
| | GE Healthcare |
| Acrodisc 13 mm Syringe filter with 0.2 | Pall Corporation |
| micron HT TUFFRYN membrane | (East Hills, NY) |
| BCA Protein Assay Kit | Pierce |
| Bovine gamma globulin | Pierce |
| Controlled Protein—Protein Cross-linking | Pierce |
| Kit | |
| N-ethylmaleimide (NMI) | Pierce |
| Alexa 488 | Molecular Probes, Inc., |
| | Carlsbad, CA |

Thiolation of Antibodies:

The antibody is adjusted to a concentration of 5 to 10 mg/mL in PBS, pH 7.4 containing 5 mM EDTA. A 5 mg/mL solution of 2-IT is prepared by dissolving it in PBS, pH 7.4 containing 5 mM EDTA. The 2-IT solution is slowly added to the antibody solution at the desired molar excess while mixing, and incubated for one hour at room temperature. The thiolated antibody is purified by applying the mixture to a desalting column equilibrated with PBS, pH 7.2 containing 5 mM EDTA. 1 mL fractions are collected and the fractions containing thiolated antibody, determined by measuring the absorbance at a wavelength of 280 nm, are pooled together. Optionally, the level of activated antibody is determined using a thiol detection reagent, (for example Ellman's reagent).

Modification of Antibodies Using Heterobifunctional Crosslinkers:

The antibody is adjusted to a concentration of 5 to 10 mg/mL in PBS, pH 7.4. The crosslinker (for example, sSMCC, SMPT, or NHS-PEO₈-Maleimide) is dissolved in DMSO to a concentration of 5 mg/mL. The crosslinker solution is slowly added to the antibody solution at the desired molar excess while mixing, and incubated for one hour at room temperature. The modified antibody is purified by applying the mixture to a desalting column equilibrated with PBS, pH 7.2 containing 5 mM EDTA. One-milliliter fractions are collected and the fractions containing modified IgG, determined by measuring the absorbance at a wavelength of 280 nm, are pooled together. Optionally, the level of activated antibody is determined using an analytical method appropriate for the crosslinker.

Conjugation of Antibodies to Photo-Reactive IRM (pIRM)

The antibody was dissolved in PBS, pH 7.4 at a concentration of 5 to 10 mg/mL. In some instances, the antibody solution was adjusted to pH 10 with 1N NaOH. The pIRM (e.g., IRM10) was dissolved in DMSO. The pIRM solution was slowly added to the antibody solution at the desired molar excess while mixing. The mixture is added to a 12-well plate (500 mL/well) and a long-wave UV light (366 nm) is placed directly over the plate for 15 minutes while on ice. The reaction was quenched by adding 1 M Tris, pH 8.0 to at 1/20 the reaction volume (v/v). The IRM-antibody conjugate was purified by size exclusion chromatography using PBS, pH 7.4 as the column running buffer at a flow rate of 1 mL/min. One-milliliter fractions were collected and the absorbance of each fraction was measured at 280 nm. Fractions containing the IRM-antibody conjugate were pooled and filtered under sterile conditions through a 0.2-micron filter. The concentration of the IRM-antibody conjugate was determined by BCA assay using bovine gamma globulin as a standard. The filtered conjugate was stored at 4° C. for future testing in biological assays.

Dye Labeled Antibodies

The antibody is adjusted to a concentration of 3 to 10 mg/mLin PBS, pH 7.4. The amine reactive dye (for example, Alexa 488) is dissolved in DMSO to a concentration of 5 mg/mL. The amine reactive dye solution is slowly added to the antibody solution at the desired molar excess (for example, 8-fold molar excess of dye to antibody) while mixing, and incubated for one hour at room temperature. The labeled antibody is purified by applying the mixture to a desalting column equilibrated with PBS, pH 7.4. 1 mL fractions are collected and the labeled antibody is pooled. The absorbance of the antibody-dye conjugate is measured at 280 nm and at the absorbance maximum for the dye (Aₘₐₓ) to determine the dye:antibody ratio. Alternately, size exclusion chromatography may be performed to purify the antibody-dye conjugate. The fractions containing dye labeled antibody are pooled together.

### Preparation of IRM-Antibody Conjugates

Conjugation of Thiolated Antibodies to Pyridyl Disulfide Modified IRM (pdIRM)

The pdIRM (e.g., IRM2) is dissolved in DMSO at a concentration of 10 mg/mL. The pdIRM is added to the thiolated antibody, as prepared above, at one half the molar excess of 2-IT used to thiolate the antibody. The mixture is incubated overnight at room temperature. To quench the reaction, a 500 mM L-cysteine solution (dissolved in 1 M Tris, pH 8.0) is added at 0.01 times (v/v) the reaction mixture. The IRM-antibody conjugate is purified by size exclusion chromatography using PBS, pH 7.4 as the column running buffer at a flow rate of 1 mL/min. 1 mL fractions are collected and the absorbance is measured at a wavelength of 280 nm. Fractions containing the IRM-antibody conjugate are pooled and filtered under sterile conditions through a 0.2-micron filter. The concentration of the IRM-antibody conjugate is determined by BCA assay using bovine gamma globulin as a standard. The filtered conjugate is stored at 4° C. for future testing in biological assays.

Conjugation of Antibodies Modified with Heterobifunctional Crosslinkers to sulfhydryl Modified IRMs (sIRM)

The sIRM (e.g., IRM1) is dissolved in DMSO at a concentration of 10 mg/mL. The sIRM is added to the modified antibody, as prepared above, at a four-fold molar excess of sIRM to the amount of crosslinker used to modify the antibody. The mixture is incubated overnight at room temperature. To quench the reaction, a 500 mM L-cysteine solution (dissolved in 1 M Tris, pH 8.0) is added at 0.01 times (v/v) the reaction mixture. The IRM-antibody conjugate is purified by size exclusion chromatography using PBS, pH 7.4 as the column running buffer, at a flow rate of 1 mL/min. 1 mL fractions of mixture are collected and measured at 280 nm. Fractions containing the IRM-antibody conjugate are pooled and filtered under sterile conditions through a 0.2-micron filter. The concentration of the IRM-antibody conjugate is determined by BCA assay using bovine gamma globulin as a standard. The filtered conjugate is stored at 4° C. for future testing in biological assays.

### Example 1

IRM1, IRM2, IRM10, CC1 and CC2 were conjugated to a human anti-CD20 antibody (RITUXAN, Genentech, San Francisco, Calif.). Conjugates prepared with IRM1 and CC1 used the general methods described above for conjugation of antibodies modified with heterobifunctional crosslinkers to sulfhydryl modified IRMs (sIRM). Specifically, the SMPT crosslinker was mixed with the antibody at a 12-fold molar excess of SMPT to antibody. Conjugates prepared with IRM2 and CC2 used the general methods described above for conjugation of thiolated antibodies to pyridyl disulfide modified IRM (pdIRM). Specifically, the 2-IT was mixed with the antibody at a 60-fold molar excess of 2-IT to antibody. The antibody conjugate prepared with IRM10 used the photo-reactive methods described in the above general method. Specifically, IRM10 was mixed with the antibody at a 20-fold (20×) and 40-fold (40×) molar excess of IRM10 to antibody.

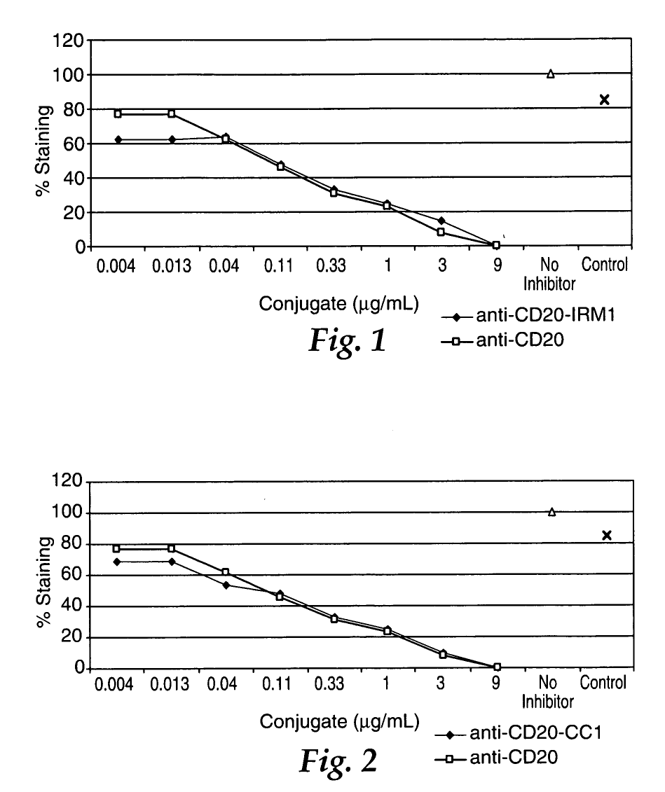

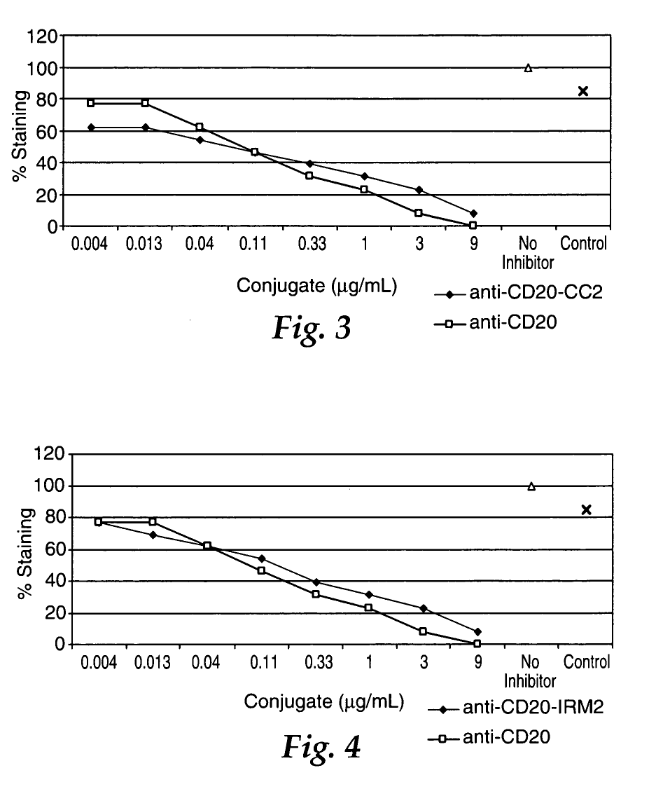

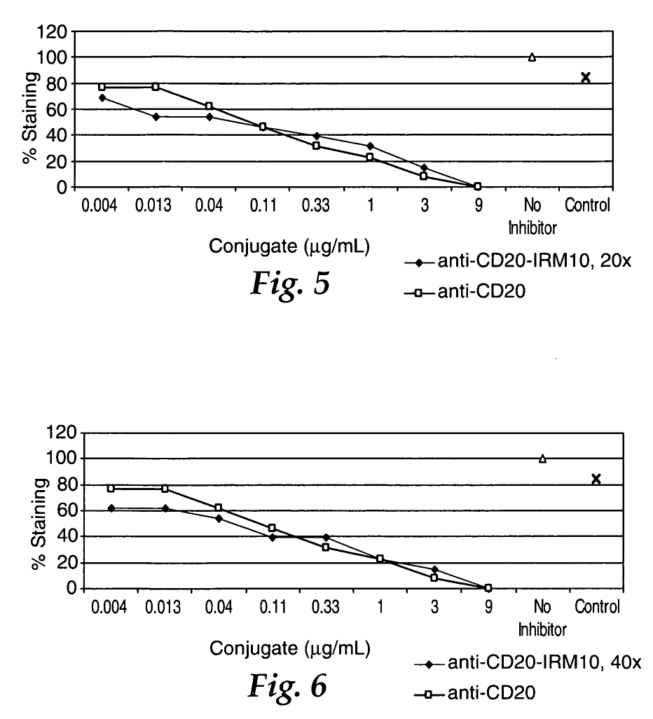

Whole blood from healthy human donors is collected by venipuncture into EDTA containing tubes. Peripheral blood mononuclear cells (PBMC) are separated from whole blood by density gradient centrifugation using HISTOPAQUE-1077 or Ficoll-Paque Plus. The PBMC layer is collected and washed twice with DPBS and resuspended in flow cytometry staining buffer (FACS buffer, Biosource). The PBMCs were added to a 96-well flat bottom sterile tissue culture plate (Costar, Cambridge, Mass. or Becton Dickinson Labware, Lincoln Park, N.J.) to a final PBMC concentration of 1×10⁶ cells/well. Anti-CD20 or the above prepared IRM/anti-CD20 antibody conjugates were added to each well at three fold dilutions from 9 μg/mL to 0.004 ug/mL, final concentration in combination with FcR blocking reagent (BD Pharmigen, San Diego, Calif.). The plate was incubated on ice for 15 minutes and then treated with 0.3-μg/mL anti-CD20-Alexa 488 per well. An Isotype (IgG1) negative control (15 μg/mL, Control, BD Pharmigen) and 0.3 μg/mL anti-CD20-Alexa 488 were placed in individual wells. The plate was incubated for 30 minutes on ice in the dark. The plate was centrifuged for 10 minutes at 1350 rpm, and cells were resuspended and washed with FACS buffer twice, resuspended in 200 μL FACS buffer and filtered through a multi-well filter plate (Pall Corporation). Cells were resuspended in 100 μL CYTOFIX buffer (BD Pharmingen) for 15 minutes at room temperature in the dark. Samples were stored overnight at 4° C. and run on a FACSCalibur (Becton Dickenson) the following day. Antibody activities of the conjugates are shown in FIGS. 1 through 6. Activity was measured by the conjugates ability to inhibit the anti-CD20-Alexa 488 to bind to the cells.

### Example 2

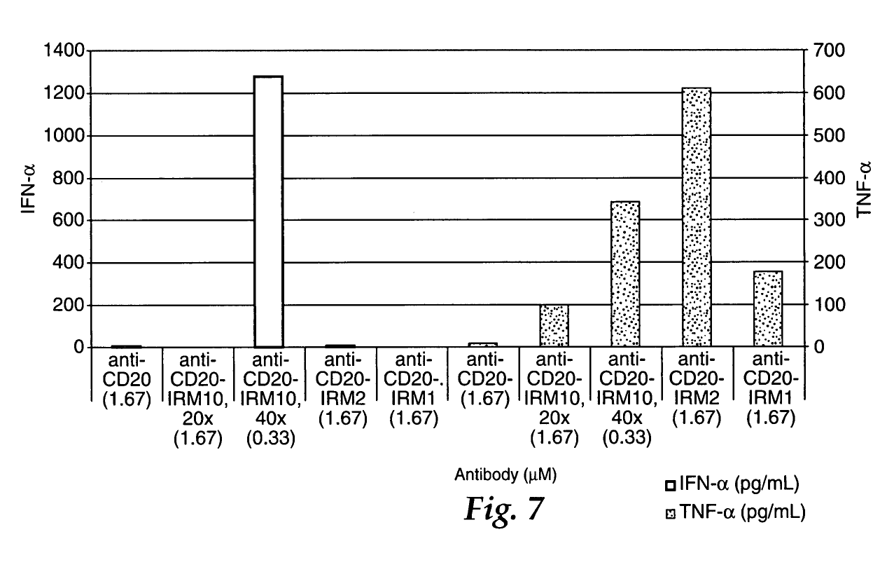

Whole blood from healthy human donors is collected by venipuncture into EDTA containing tubes. Peripheral blood mononuclear cells (PBMC) are separated from whole blood by density gradient centrifugation using HISTOPAQUE-1077 or Ficoll-Paque Plus. The PBMC layer is collected and washed twice with DPBS or HBSS and resuspended at 4×10⁶ cells/mL in RPMI complete media. The PBMCs were added to a 96-well flat bottom sterile tissue culture plate (Costar, Cambridge, Mass. or Becton Dickinson Labware, Lincoln Park, N.J.) to a final PBMC concentration of 2×10⁶ cells/mL. PBMCs were stimulated overnight at 37° C. in a 5% carbon dioxide atmosphere with 0.33 μM, 0.420 μM, 0.830 μM, or 1.67 μM anti-CD20 or the anti-CD20 conjugates prepared in Example 1, based on final antibody concentration. Culture supernatants were analyzed for IFN-α and TNF production using a human IFN-α ELISA (PBL Biomedical Laboratories, Piscataway, N.J.) and human-specific TNF BV™ immunoassay (BioVeris Corp., Gaithersburg, Md.), respectively, with results expressed in pg/mL. Cytokine induction by the conjugates is shown in FIG. 7.

### Example 3

IRM2 and IRM10 were conjugated to a mouse anti-CD40 antibody (FGK4.5). The method used to conjugate anti-CD40 to IRM2 was the same as described in Example 1; however, a 70-fold molar excess of 2-IT to anti-CD40 was used in the preparation of the antibody conjugate. The method used to conjugate anti-CD40 to IRM10 was the same as described in Example 1; however, an 8-fold (8×) or 25-fold (25×) molar excess of IRM10 to anti-CD40 was used in the preparation of these antibody conjugates.

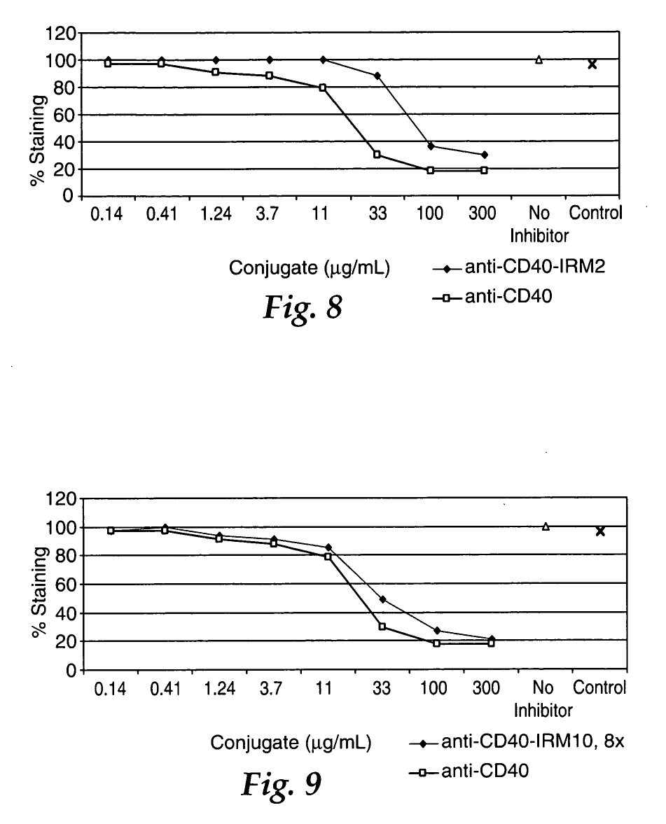

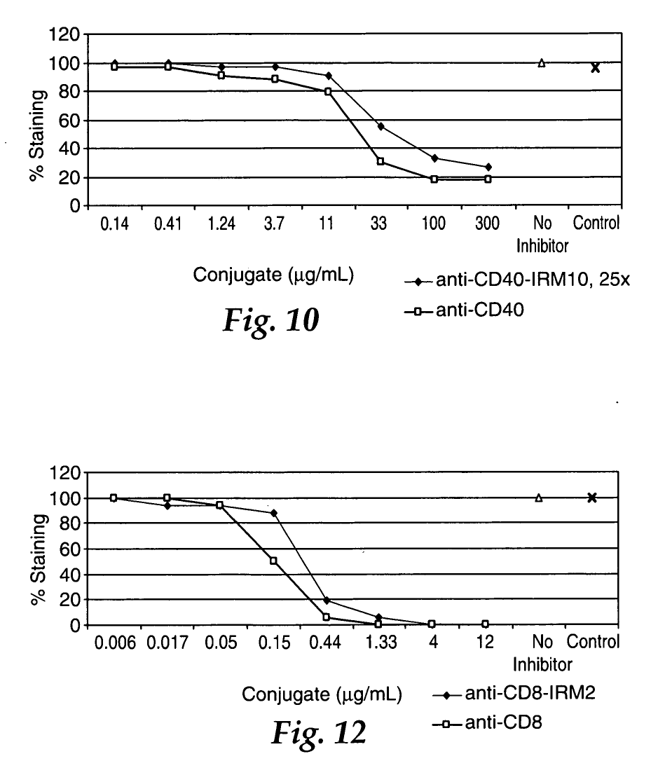

Mouse spleens were removed from sacrificed C57BL6 mice and splenocytes were isolated from the mice by homogenizing the spleens. Splenocytes were homogenized in Hanks Balance Salt Solution media (Biosource International, Camarillo, Calif.) containing 1% FCS, washed and resuspended in FACS buffer (Biosource International). Splenocytes were plated in a 96-well round bottom sterile tissue culture plate (Costar, Cambridge, Mass. or Becton Dickinson Labware, Lincoln Park, N.J.) to a final cell concentration of 2×10⁶ cells/well. Anti-CD40 or the above-prepared conjugate were added to each well at three fold dilutions from 300 μg/mL to 0.14 μg/mL, final concentration in combination with mouse FcR blocking reagent (2.4G2). The plate was incubated on ice for 15 minutes and then treated with 25-ug/mL anti-CD40-Alexa 488 per well. An Isotype (IgG2a) negative control (15 μg/mL, Control, BD Pharmigen) and 25 μg/mL anti-CD40-Alexa 488 were placed in individual wells. The plate was incubated for 30 minutes on ice in the dark. The plate was centrifuged for 10 minutes at 1500 rpm, and cells were resuspended and washed with FACS buffer twice, resuspended in 100 μL CYTOFIX buffer (BD Pharmigen) for 15 minutes at room temperature in the dark. Cells were washed and resuspended in 200 μL FACS buffer and filtered through a Multi-well filter plate. Samples were stored overnight at 4° C. and run on a FACSCalibur (Becton Dickenson) the following day. Antibody activities of the conjugates are shown in FIGS. 8 through 10. Activity was measured by the conjugates ability to inhibit the anti-CD40-Alexa 488 to bind to the cells.

### Example 4

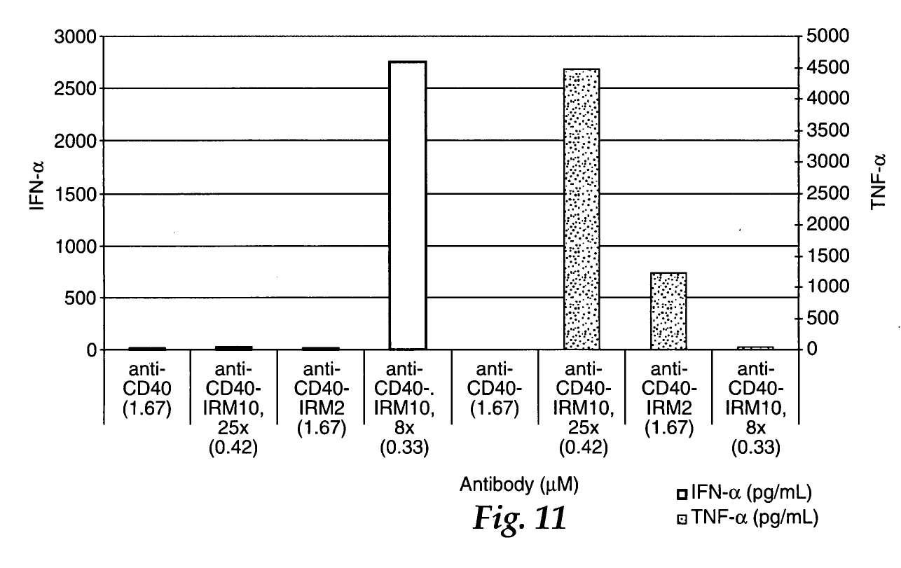

The anti-CD40 antibody and conjugate, as prepared in Example 3, were tested for cytokine induction as described in Example 2. Cytokine induction by the conjugate is shown in FIG. 11.

### Example 5

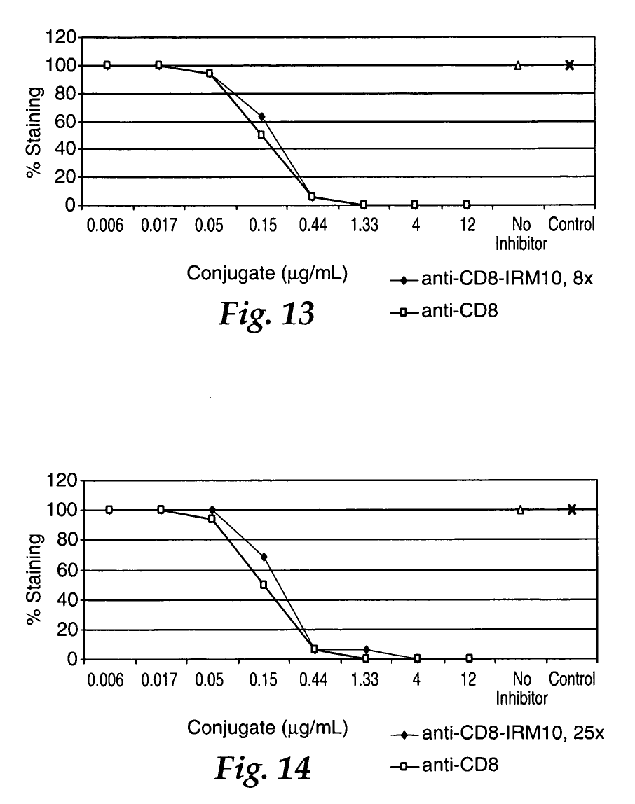

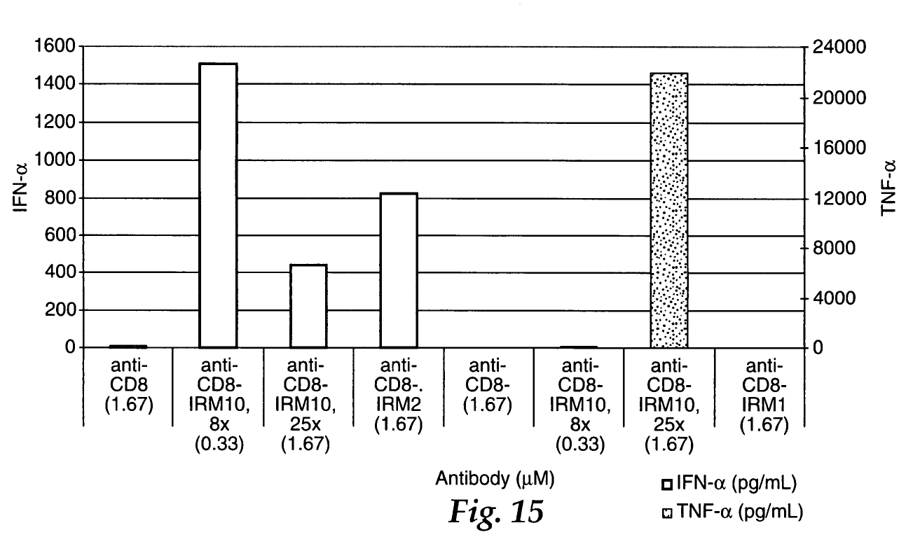

IRM2 and IRM10 were conjugated to a mouse anti-CD8 antibody (53.6.72; ATCC, Manassas, Va.). The methods used to conjugate anti-CD8 to IRM2 or IRM10 were the same as those described in Example 3. The conjugate was tested for antibody activity, as described in Example 3, using three fold dilutions of the conjugate from 12 μg/mL to 0.006 μg/mL and 0.40 μg/mL for the anti-CD8-Alexa 488. The anti-CD8 antibody and conjugates were also tested for cytokine induction as described in Example 2. Antibody activities of the conjugates are shown in FIGS. 12 through 14. Activity was measured by the conjugates ability to inhibit the anti-CD8-Alexa 488 to bind to the cells. Cytokine induction by the conjugate is shown in FIG. 15.

### Example 6

IRM10 was conjugated to the HERCEPTIN antibody (HER2; Genentech) using the same method that was described in Example 1 except that a 28.4-fold (pH 7.4), 28.4-fold (pH 10), or 42.6-fold (pH 10) molar excess of IRM10 to HER2 was used in the preparation of the antibody conjugate.

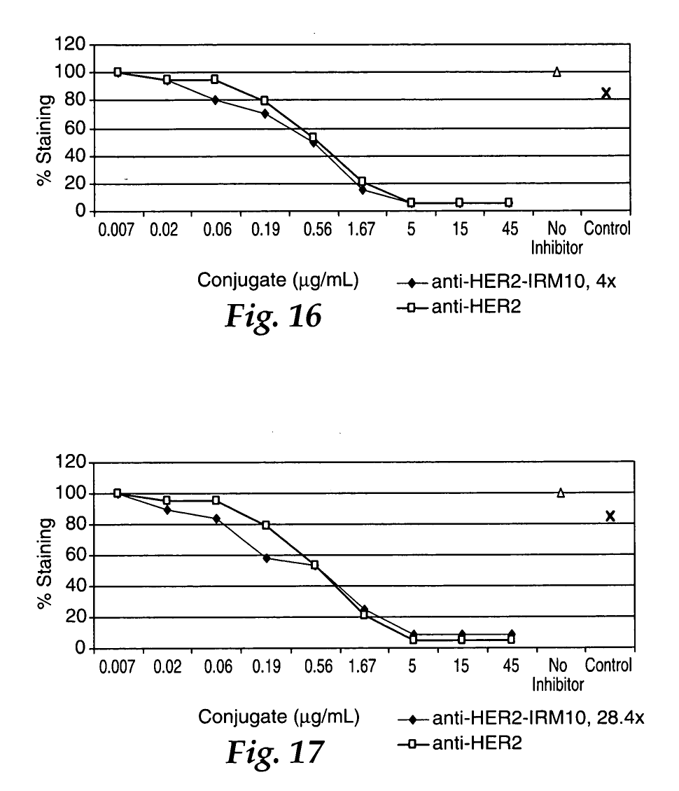

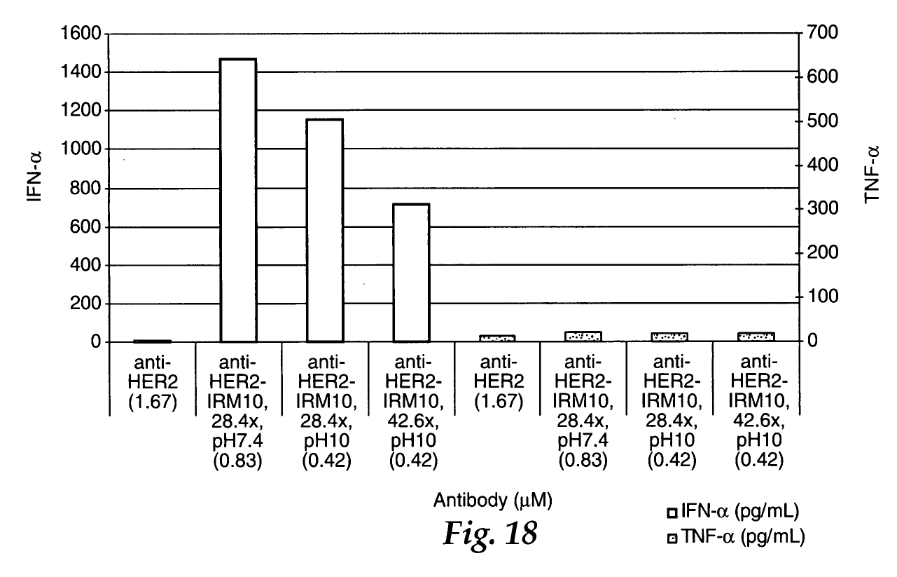

The HER2 antibody and conjugate were tested for antibody activity as described in Example 2; however, her2 positive human breast cancer cells (HCC2218; ATCC, Manassas, Va.) were used in the assay (2.5×10⁵ cells/well) instead of human PBMCs. The conjugate was tested for antibody activity using three fold dilutions of the conjugate from 45 μg/mL to 0.007 μg/mL and 0.30 μg/mL for the HER2-Alexa 488. Antibody activities of the conjugates are shown in FIGS. 16 and 17. The HER2 antibody and conjugate were tested for cytokine induction as described in Example 2. Cytokine induction by the conjugate is shown in FIG. 18.

### Example 7

IRM1 was conjugated to HER2 as described in Example 1; however, a NHS-PEO₈-Maleimide crosslinker was mixed with the antibody at a 15-fold molar excess of NHS-PEO₈-Maleimide to antibody.

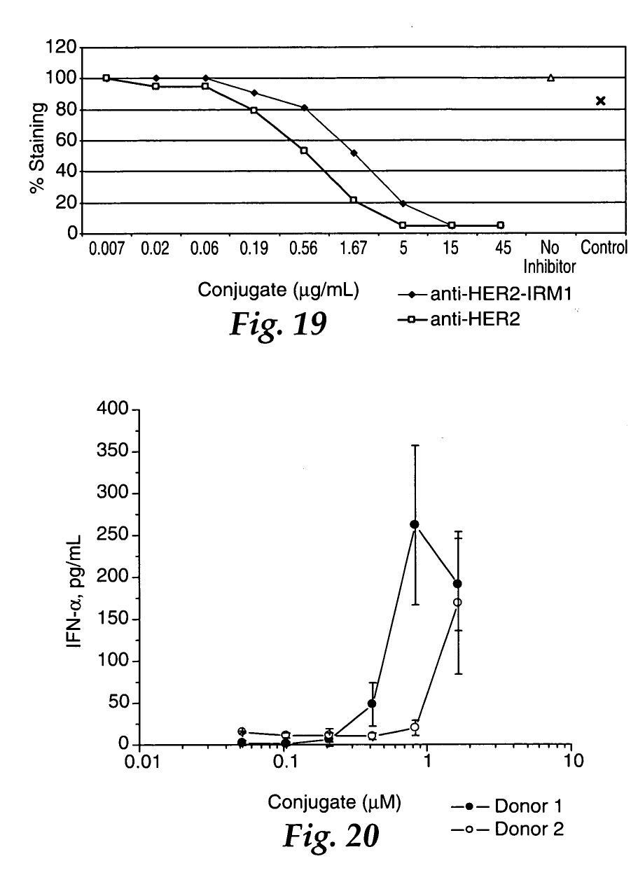

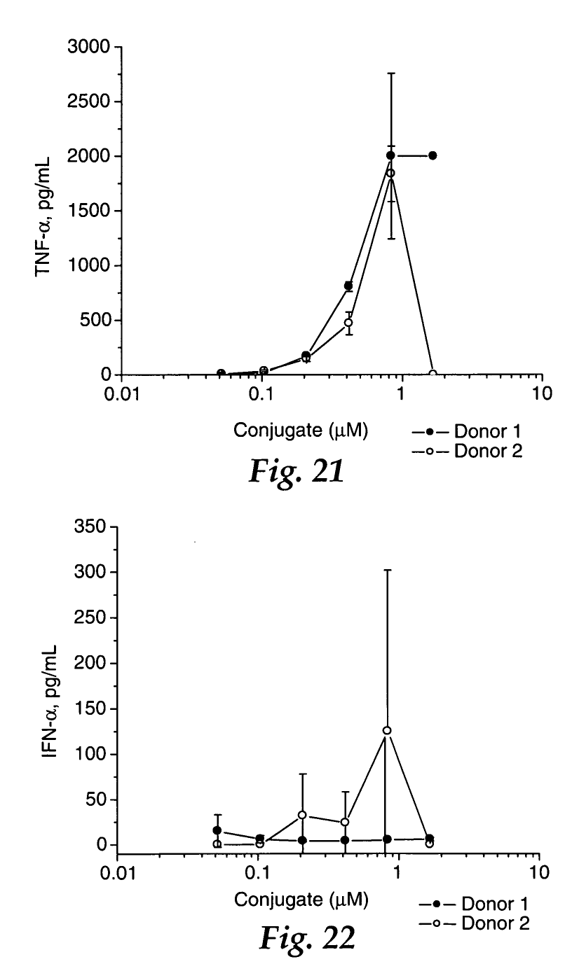

The HER2 antibody and conjugate were tested for antibody activity as described in Example 6. Activity was measured by the conjugates ability to inhibit the HER2-Alexa 488 to bind to the cells. Antibody activities of the conjugates are shown in FIG. 19. The HER2 antibody and conjugate were tested for cytokine induction as described in Example 2. Cytokine induction by the conjugate is shown in FIGS. 20 and 21.

### Example 8

IRM1 was conjugated to the anti-CD8 antibody as described in Example 1; however, a NHS-PEO₈-Maleimide crosslinker was mixed with the antibody at a 15-fold molar excess of NHS-PEO₈-Maleimide to antibody.

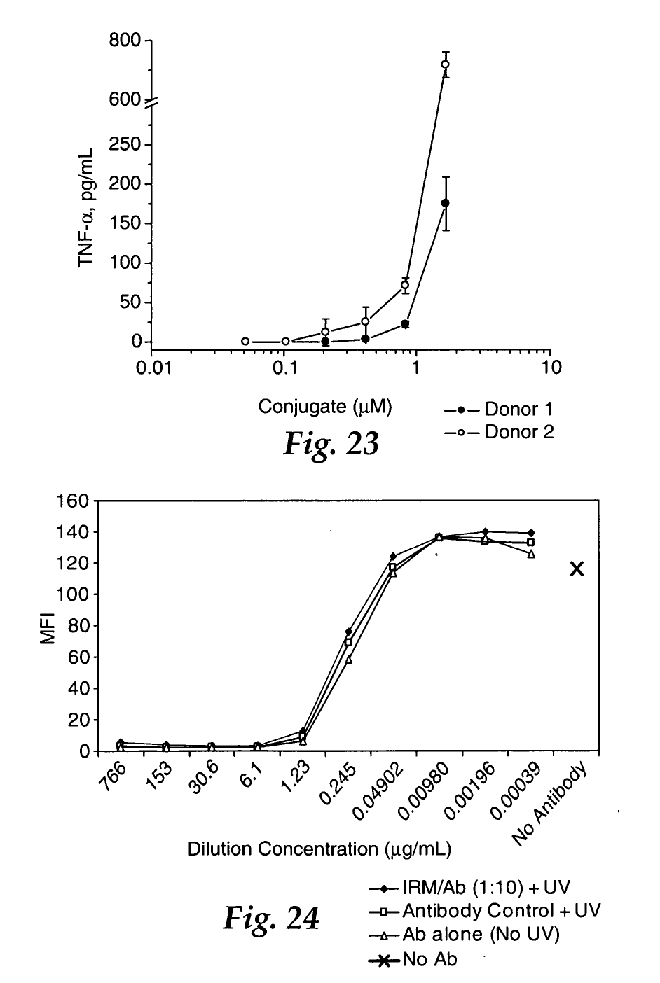

The anti-CD8 antibody and conjugate were tested for cytokine induction as described in Example 2. Cytokine induction by the conjugate is shown in FIGS. 22 and 23.

### Example 9

IRM10 was suspended in dimethyl sulfoxide (DMSO) to 10 mg/mL. Rat anti-mouse CD8 antibody (53.6.72, BioExpress, Inc., West Lebanon, N.H.) was suspended in phosphate buffered saline (PBS) to 7.8 mg/mL and the pH adjusted to >9.0 by the addition of NaOH. A 1:10 ratio of IRM:antibody (volume:volume) was mixed together by adding 60 μL of the IRM10 solution (0.6 mg IRM1IRM10) with 540 μL of the anti-CD8 antibody solution (4.21 mg anti-CD8). The antibody control was 60 μL of PBS mixed with 540 μL of the anti-CD8 antibody solution. The 1:10 IRM:antibody, 1:100 IRM:antibody, and antibody control were each placed in single wells of a 24-well tissue culture plate. The plate was placed on ice and a long wavelength UV light source was placed directly over the plate as close to the well containing the IRM10/antibody mixture as possible. The mixtures were irradiated for 15 minutes. The resulting conjugate and antibody control were removed from the wells and resuspended in PBS to a final concentration of 0.5 mg/mL IRM10, 3.51 mg/mL anti-CD8; 0.05 mg/mL IRM10, 3.86 mg/mL anti-CD8; and 3.51 mg/mL anti-CD8 for the 1:10, 1:100, and antibody control, respectively, and dialyzed using a 10,000 molecular weight cutoff Slide-a-Lyzer (Pierce, Rockford, Ill.) against PBS to remove any unconjugated IRM.

### Example 10

Mouse spleens were removed from sacrificed C57BL6 mice and splenocytes were isolated from the mice by homogenizing the spleens. Splenocytes were homogenized in EHAA media (Biosource International, Camarillo, Calif.) containing 1% FCS, washed and resuspended in FACS buffer (Biosource International). Splenocytes were plated in a 96 well round bottom sterile tissue culture plate (Costar, Cambridge, Mass. or Becton Dickinson Labware, Lincoln Park, N.J.) to a final cell concentration of 1×10⁶ cells/well.

Splenocytes were treated for 30 minutes at 4° C. with the IRM:antibody (1:10), antibody control, both as prepared in Example 9, or non-UV treated antibody at five fold dilution concentrations ranging from 766 μg/mL to 0.002 μg/mL. After the 30-minute treatment time, FITC-labeled mouse anti-CD8 (53.6.7, BD Pharmingen, San Diego, Calif.) and PE-labeled mouse anti-CD3 (BD Pharmingen) were added to all the wells and incubated for 30 minutes at 4° C. Cells were then washed two times with FACS buffer and fixed with CYTOFIX buffer (BD Pharmingen, San Diego, Calif.). Flow cytometry analysis was performed by gating on the CD3+ lymphocytes and measuring the mean fluorescence intensity (MFI) of the FITC-labeled antibody. Results are shown in FIG. 24.

### Example 11

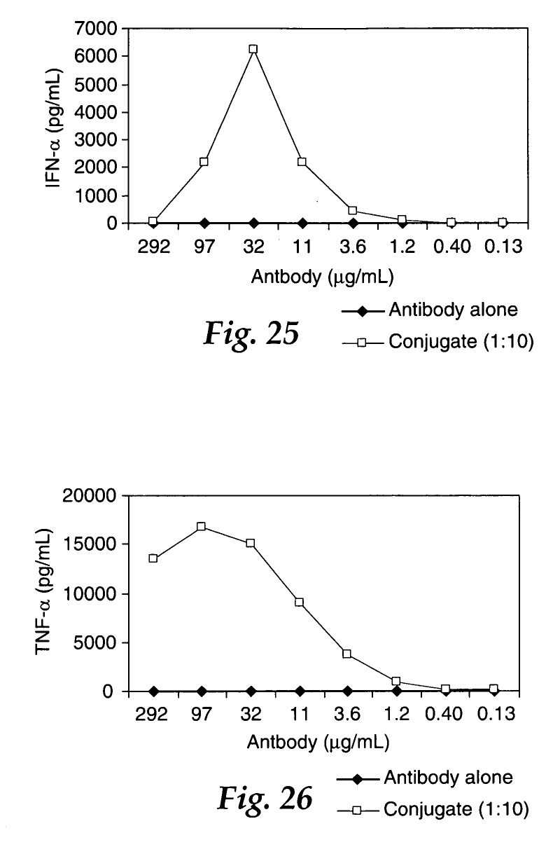

Whole blood from healthy human donors was collected by venipuncture into EDTA vacutainer tubes. Peripheral blood mononuclear cells (PBMC) were separated from whole blood by density gradient centrifugation using Histopaque®-1077. Blood was diluted 1:1 with Dulbecco's Phosphate Buffered Saline (DPBS) or Hank's Balanced Salts Solution (HBSS). The PBMC layer was collected and washed twice with DPBS or HBSS and resuspended at 4×10⁶ cells/mL in RPMI complete media. The PBMCs were added to a 96 well flat bottom sterile tissue culture plate (Costar, Cambridge, Mass. or Becton Dickinson Labware, Lincoln Park, N.J.) to a final PBMC concentration of 2×10⁶ cells/mL. PBMCs were stimulated overnight at 37° C. in a 5% carbon dioxide atmosphere with the rat anti-mouse CD8 antibody alone or the 1:10 IRM/antibody conjugate as described in Example 9 in 3-fold antibody dilutions (292-0.13 μg/mL). Culture supernatants were analyzed for IFN-α and TNF production using a human IFN-α ELISA (PBL Biomedical Laboratories, Piscataway, N.J.) and human-specific TNF BV™ immunoassay (BioVeris Corp., Gaithersburg, Md.), respectively, with results expressed in pg/mL. Results are shown in FIG. 25 and FIG. 26.

The complete disclosures of the patents, patent documents, and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

What is claimed is:

1. An immunomodulatory composition comprising:
an IRM moiety coupled to a targeting moiety.

2. The immunomodulatory composition of claim 1 wherein the IRM moiety is an agonist of at least one TLR.

3. The immunomodulatory composition of claim 1 further comprising a spacer arm or solid support to which the IRM moiety and the targeting moiety are attached.

4. The immunomodulatory composition of claim 1 wherein the spacer arm length is from about 20 Å to about 100 Å.

5. The immunomodulatory composition of claim 1 wherein the solid support comprises a particle having a diameter of from 1 nm to about 200 nm.

6. The immunomodulatory composition of claim 1 wherein IRM moiety and the targeting moiety are affinity coupled.

7. The immunomodulatory composition of claim 1 wherein IRM moiety and the targeting moiety are covalently coupled.

8. The immunomodulatory composition of claim 1 wherein the targeting moiety recognizes at least a portion of a tumor-specific antigen or marker.

9. The immunomodulatory composition of claim 8 wherein the targeting moiety recognizes at least a portion of at least one antigen or marker specific for breast cancer, colon cancer, pancreatic cancer, prostate cancer, lung cancer, prostate cancer, liver cancer, or melanoma.

10. The immunomodulatory composition of claim 1 wherein the targeting moiety comprises a ligand of a tumor-specific marker.

11. The immunomodulatory composition of claim 10 wherein the targeting moiety comprises a Leuteinizing hormone releasing hormone (LHRH) receptor ligand.

12. The immunomodulatory composition of claim 10 wherein the targeting moiety comprises a folic acid receptor ligand.

13. The immunomodulatory composition of claim 1 wherein the targeting moiety comprises bis-phosphonate.

14. The immunomodulatory composition of claim 1 wherein the targeting moiety recognizes at least a portion of at least one endothelial antigen or marker.

15. The immunomodulatory composition of claim 1 wherein the targeting moiety recognizes at least a portion of a dendritic cell surface antigen or marker.

16. The immunomodulatory composition of claim 1 wherein the targeting moiety recognizes at least a portion of a surface antigen or marker of a cell that, when activated, is capable of killing a tumor cell.

17. The immunomodulatory composition of claim 16 wherein the targeting moiety recognizes at least a portion of a surface antigen or marker of a cytotoxic T lymphocyte, an NKT cell, or an NK cell.

18. The immunomodulatory composition of claim 1 further comprising a second targeting moiety.

19. The immunomodulatory composition of claim 18 wherein one target specific moiety recognizes at least a portion of an antigen or marker specific for an immune cell and the second targeting moiety recognizes an antigen or marker specific for a tumor cell.

20. The immunomodulatory composition of claim 18 wherein one target specific moiety recognizes at least a portion of an antigen or marker specific for an immune cell and the second targeting moiety recognizes at least a portion of an endothelial antigen or marker.

21. A method of targeted delivery of an IRM compound, the method comprising:
administering to a subject an immunomodulatory composition that includes an IRM moiety coupled to a targeting moiety that recognizes a delivery target.

22. The method of claim 21 wherein the delivery target comprises a tumor cell.

23. The method of claim 21 wherein the delivery target comprises an immune cell.

24. A method of inducing a localized immune response, the method comprising:
administering to a subject an immunomodulatory composition that includes an IRM moiety coupled to a targeting moiety that recognizes a delivery target in an amount effective to induce an immune response.

25. The method of claim 24 wherein the delivery target comprises a tumor cell and the immune response is directed against the delivery target.

26. The method of claim 24 wherein the delivery target is an immune cell and the immune response is at least partially generated by the delivery target.

27. A method of treating a condition in a subject that is treatable by inducing an immune response, the method comprising:
administering to the subject an immunomodulatory composition that includes an IRM moiety coupled to a targeting moiety that recognizes a delivery target in an amount effective to treat at least one symptom or sign of the condition.

28. The method of claim 27 wherein the amount effective to treat at least one symptom or sign of the condition is an amount effective to ameliorate at least one symptom or sign of the condition.

29. The method of claim 27 wherein the amount effective to treat at least one symptom or sign of the condition is an amount effective to reduce an increase of at least one symptom or sign of the condition.

30. An immunomodulatory composition comprising:
an IRM moiety coupled to a targeting moiety, wherein the IRM moiety is a compound of the formula: 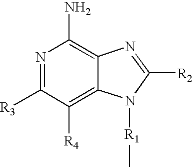
wherein:
R₁ is a linker group;
R₂ is selected from the group consisting of:
-hydrogen;
-alkyl;
-alkenyl;
-aryl;
-substituted aryl;
-heteroaryl;
-substituted heteroaryl;
-alkyl-O-alkyl;
-alkyl-S-alkyl;
-alkyl-O-aryl;
-alkyl-S-aryl:
-alkyl-O-alkenyl;
-alkyl-S-alkenyl; and
-alkyl or alkenyl substituted by one or more substituents selected from the group consisting of:
-OH;
-halogen;
—N(R₅)₂;
—CO—N(R₅)₂;
—CS—N(R₅)₂;
—SO₂—N(R₅)₂;
—NR₅—CO—C₁₋₁₀ alkyl;
—NR₅—CS—C₁₋₁₀ alkyl;
—NR₅—SO₂-C₁₋₁₀ alkyl;
—CO—C₁₋₁₀ alkyl;
—CO—O-C₁₋₁₀ alkyl;
—N₃;
-aryl;
-substituted aryl;
-heteroaryl;
-substituted heteroaryl;
-heterocyclyl;
-substituted heterocyclyl;
—CO-aryl;
—CO-(substituted aryl);
—CO-heteroaryl; and
—CO-(substituted heteroaryl);
R₃ and R₄ are each independently:
-hydrogen;
-halogen;
-alkyl;
-alkenyl;
—O-alkyl;
—S-alkyl; and
—N(R₅)₂;
or when taken together, R₃ and R₄ form a fused aryl or heteroaryl group that is optionally substituted by one or more substituents selected from the group consisting of;
-halogen;
-alkyl;
-alkenyl;
—O-alkyl;
—S-alkyl; and
—N(R₅)₂;
or when taken together, R₃ and R₄ form a fused 5 to 7 membered saturated ring, optionally containing one or more heteroatoms and optionally substituted by one or more substituents selected from the group consisting of,
-halogen;
-alkyl;
-alkenyl;
—O-alkyl;
—S-alkyl; and
—N(R₅)₂; and
each R₅ is independently hydrogen or C₁₋₁₀ alkyl.
